(19) United States
(12) Patent Application Publication
(10) Pub. No.: US 2024/0286996 A1
(43) Pub. Date: Aug. 29, 2024

(54) NAPHTHALENESULFONYL COMPOUNDS, AND PREPARATION METHODS AND APPLICATIONS

(71) Applicant: FUDAN UNIVERSITY, Shanghai (CN)

(72) Inventors: Huiru TANG, Shanghai (CN); Pengcheng LI, Shanghai (CN)

(73) Assignee: FUDAN UNIVERSITY, Shanghai (CN)

(21) Appl. No.: 18/434,874

(22) Filed: Feb. 7, 2024

Related U.S. Application Data
(63) Continuation of application No. PCT/CN2022/111727, filed on Aug. 11, 2022.

(30) Foreign Application Priority Data
Aug. 11, 2021 (CN) 202110916773.4

(51) Int. Cl. C07C 309/51; C07B 59/00; C07C 309/88; G01N 30/02; G01N 30/06; G01N 30/08

(52) U.S. Cl. CPC C07C 309/51; C07B 59/001; C07C 309/88; G01N 30/08; C07B 2200/05; G01N 2030/027; G01N 2030/067

(57) ABSTRACT

The present disclosure provides naphthalenesulfonyl compounds, preparation methods and application thereof. Specifically disclosed is a compound of formula (I) or a salt thereof, which serves as a specific derivatization reagent capable of reacting with hydroxyl and amino groups. The compound features simple synthesis, high reactivity, and ready availability at low cost, and is capable of improving chromatographic separation behaviors of target compounds and enhancing the detection sensitivities of these compounds.

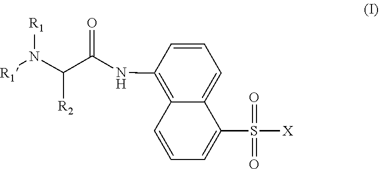

### CROSS-REFERENCE TO RELATED APPLICATIONS

This application is a continuation of International Application No. PCT/CN2022/111727, filed on Aug. 11, 2022, which claims priority to Chinese Patent Application No. 202110916773.4, filed on Aug. 11, 2021. The disclosures of the aforementioned applications are hereby incorporated by reference in their entireties.

### TECHNICAL FIELD

The present disclosure relates to naphthalenesulfonyl compounds, and preparation methods and applications thereof.

### BACKGROUND

Chemical labeling, i.e., stable isotope coded derivatization (ICD), is a technique of introducing mass difference tags in the form of light-labeled and heavy-labeled isotopes into a target for relative quantitative analysis. The labeling technique is applicable to quantitative analysis of target components in complex matrix samples. Where the concentration of one group of samples is known, analytes in the sample may be absolutely quantified by this technique.

The chemical labeling technique was applied to quantitative analysis of proteome in early stages. With the development of metabonomics, the stable isotope labeling technique was gradually applied to highly sensitive detection of important small molecule metabolites such as amines, aldehydes and ketones, carboxylic acid metabolites, and the like.

Appropriate derivatization reagents need to be selected in compliance with requirements: (1) The derivatization reagent is easy to synthesize, and isotopic labeling in the derivatization reagent is achieved at a lower cost; (2) specific derivatization labeling is achieved for target functional groups, and the reaction efficiency is stable; (3) the derivatization reaction conditions are mild and do not destroy the existing form of endogenous target compounds in the system; (4) the derivatized product is effectively ionized for MS detection; and (5) the isotope effect is small, and there is basically no retention time drift.

In 1999, Gygi et al. developed the technique (reagent) of mass difference tagging—isotope coded affinity tagging (ICAT). The reagent mainly included three parts: an affinity tag composed of biotin, a linker group for introducing a stable isotope, and a reactive group for specifically binding to a sulfhydryl group of a cysteine residue in a peptide segment. In 2005, Che et al. designed a labeling reagent, 4-trimethylammoniumbutyryl amide (TMAB), which was used for all amino-containing substances, and used the reagents labeled with D and H separately to achieve quantitative analysis. Multiple deuterated labeling sites on TMAB and ICAT reagents result in a significant isotope effect, which affects the retention time of labeled analytes on a chromatographic column.

In 2003, Thompson et al. synthesized isobaric tags, known as TMT tags (tandem mass tags). TMT includes four parts: mass reporter region, cleavable linker region, mass balance region, and an amino reactive group. The unique structure of the TMT reagent enables different isotopically labeled forms of the target molecule to have identical chromatographic behaviors and first order MS characteristics. By secondary mass spectrometry scanning, amino compounds with different labeling forms are fragmented in the cleavable region to form different reporter ions, and the relative content change of the sample is determined by comparing the intensities of the reporter ions. The TMT reagent is mainly labeled with ¹³C, and the synthesis is cumbersome, costly and low-yielding, and thus the use of this reagent is greatly limited.

In 2004, Applied Biosystems proposed isobaric tags for relative and absolute quantification (iTRAQ) technology that is identical to the TMT labeling strategy. By changing the isotopic number and species of the equilibrium reporter group and the equilibrium group, which are designed as four labeling modes with the same molecular weight but different reporter groups, the four groups of biological samples can be isotopically labeled at the same time, and the target in multiple samples can be quantitatively analyzed using the reporter ion response of the reporter group in MS/MS. Currently, iTRAQ has been developed to an eight-fold labeled reagent, but it is costly and susceptible to interference from amino-containing species in the sample.

With the stable isotope labeling technique based on chemical derivatization, the mass difference functional group with isotope can be labeled on different biological samples, such that the light-labeled/heavy-labeled isotope tags reflecting the information of samples can be obtained, and then the quantitative information of different metabolites can be obtained by comparing the mass response differences of the light-labeled and heavy-labeled target components using liquid chromatography-mass spectrometry. This technique has been widely applied to the common metabolites of amines, hydroxyls, phenolic hydroxyls, carboxylic acids and aldehydes and ketones, which provides novel ideas and strategies for derivatization-assisted mass spectrometry analysis of nucleoside metabolites.

### SUMMARY

The present disclosure is intended to address the defects of the conventional specific derivatization reagents, such as high price, severe isotope effect, poor detection sensitivity, complicated synthetic steps, and the like. Accordingly, the present disclosure provides a naphthalenesulfonyl compound, and a preparation method and application thereof. The naphthalenesulfonyl compound according to the present disclosure, as a type of specific derivatization reagents which can react with hydroxyl and amino groups, features simple synthesis, high reactivity, low cost, and ready availability at low cost, and is capable of improving chromatographic separation behaviors of target compounds, and enhancing detection sensitivities of these compounds.

The present disclosure solves the above problem by employing the following technical solution:

The present disclosure provides a compound of formula (I) or a salt thereof:

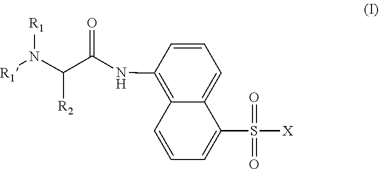

wherein R₁ and R_{1′} are independently selected from C₁₋₇ alkyl;
R₂ is selected from H, C₁₋₇ alkyl, or benzyl; and
X is selected from OH or halogen.

In an example of the present disclosure, some groups of the compound of formula (I) or the salt thereof are defined as follows, and the groups not mentioned are as described in any of the examples of the present disclosure (hereinafter referred to as “in an example of the present disclosure”). In R₁ or R_{1′}, the C₁₋₇ alkyl is C₁₋₄ alkyl, for example C₂₋₄ alkyl, still for example ethyl.

In an example of the present disclosure, in R₂, the C₁₋₇ alkyl is C₁₋₄ alkyl, for example, isobutyl.

In an example of the present disclosure, in X, the halogen is Cl.

In an example of the present disclosure, R₁ and R_{1′} are the same.

In an example of the present disclosure, R₂ is C₁₋₇ alkyl.

In an example of the present disclosure, the compound of formula (I) is

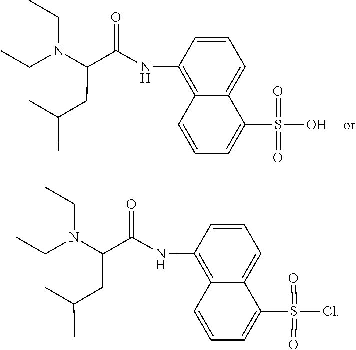

In an example of the present disclosure, the salt of the compound of formula (I) is a salt obtained from the compound of formula (I) and an acid, wherein the acid is an inorganic acid or an organic acid, and preferably the organic acid.

The present disclosure further provides a preparation method for a compound of formula (I). The preparation method includes a process I or a process II.

The process I includes: subjecting a compound of formula (III) to a condensation reaction with a compound of formula (IV) in the presence of an activating agent and a base in a solvent to obtain the compound of formula (I):

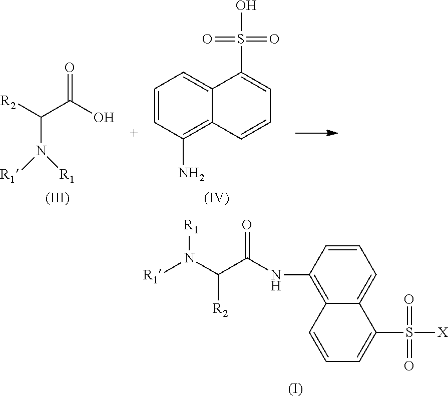

in the process (I), X is OH, and definitions of R₁, R_{1′}, and R₂ are as recited above; or

The process (II) includes:
(1) subjecting a compound of formula (III) to a condensation reaction with a compound of formula (IV) in the presence of an activating agent and a base in a solvent to obtain a compound of formula (V):

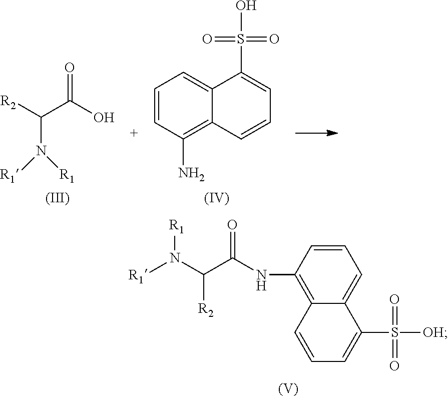

(2) subjecting the compound of formula (V) to an acylation reaction with a chlorinating agent to obtain the compound of formula (I):

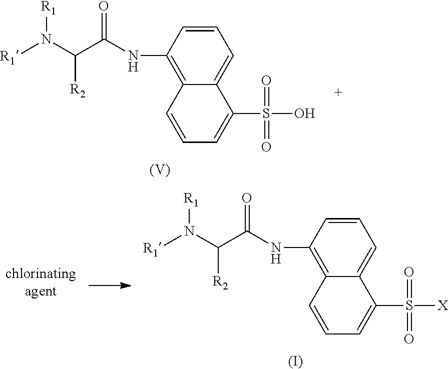

in the process (II), X is Cl, and definitions of R₁, R_{1′}, and R₂ are as recited above.

In an example of the present disclosure, the solvent is a solvent conventional in the art for such reactions, for example, N,N-dimethylformamide (DMF).

In an example of the present disclosure, in the condensation reaction, the activating agent is conventional activating agent for reactions in the field, for example, includes one or more of 4-(4,6-dimethoxytriazin-2-yl)-4-methylmorpholine hydrochloride, 1-(3-dimethylaminopropyl)-3-ethylcarbodiimide hydrochloride and 1-hydroxybenzotriazole, for example, 4-(4,6-dimethoxytriazin-2-yl)-4-methylmorpholine hydrochloride or “a combination of 1-(3-dimethylaminopropyl)-3-ethylcarbodiimide hydrochloride and 1-hydroxybenzotriazole”.

In an example of the present disclosure, in the condensation reaction, the base may is base conventional for such reactions in the art, for example, an organic base, N-methylmorpholine (NMM) and/or pyridine (Py)

In an example of the present disclosure, the condensation reaction is carried out at a temperature conventional for such reactions in the art, for example, room temperature.

In an example of the present disclosure, a progress of the condensation reaction is detected using a conventional monitoring approach in the art (e.g., TLC, HPLC, or NMR), and typically the time when the compound of formula (III) disappears or no longer reacts is taken as a reaction endpoint. A duration of the condensation reaction is in the range of 8-24 hours.

In an example of the present disclosure, in the acid chlorination reaction, the solvent is a solvent conventional for such reactions in the art, for example, tetrahydrofuran (THF) and/or toluene.

In an example of the present disclosure, in the acylation reaction, the chlorinating agent is a chlorinating agent conventional for such reactions in the art, for example, phosphorus pentachloride and/or oxalyl chloride.

In an example of the present disclosure, the acid chlorination reaction is carried out at a temperature conventional in the art for such reactions, for example, room temperature.

In an example of the present disclosure, a progress of the acid chlorination reaction is detected using a conventional monitoring approach in the art (e.g., TLC, HPLC, or NMR), and typically the time when the compound of formula (V) disappears or no longer reacts is taken as a reaction endpoint. A duration of the acylation reaction may be in the range of 5 minutes to 4 hours.

The preparation method for a compound of formula (I) further includes a process (1-1) or a process (1-2); wherein

the process (1-1) includes: subjecting a compound of formula (VI) to a reductive amination reaction with a compound of formula (A-1) and a compound of formula (A-2) in the presence of a reducing agent in a solvent to obtain the compound of formula (III):

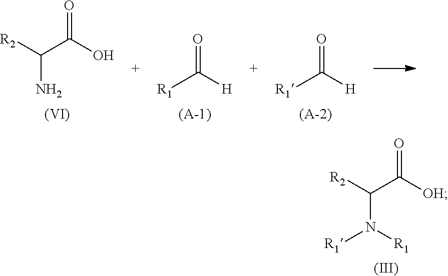

the process (1-2) includes: subjecting a compound of formula (VI) to an alkylation reaction with a compound of formula (B-1) and a compound of formula (B-2) in the presence of a base in a solvent to obtain the compound of formula (III):

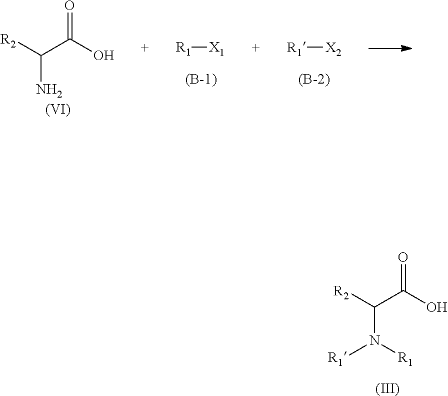

wherein X₁ and X₂ are independently halogen (for example, I); and

wherein in the processes (1-1) and (1-2), definitions of R₁, R_{1′}, and R₂ are as recited above.

In an example of the present disclosure, in the reductive amination reaction, the solvent is a solvent conventional for such reactions in the art is methanol, acetonitrile, or a buffer of sodium acetate or phosphate with a pH of 2 to 12.

In an example of the present disclosure, in the reductive amination reaction, the reducing agent is a reducing agent conventional for such reactions in the art, for example, sodium cyanoborohydride and/or 2-methylpyridine borane.

In an example of the present disclosure, the reductive amination is carried out at a temperature conventional for such reactions in the art, for example, 30° C. to 40° C.

In an example of the present disclosure, a progress of the reductive amination reaction is detected using a conventional monitoring approach in the art (e.g., TLC, HPLC, or NMR), and typically the time when the compound of formula (VI) disappears or no longer reacts is taken as a reaction endpoint. A duration of the reductive amination reaction may be in the range of 20 hours to 28 hours.

In an example of the present disclosure, the solvent is a solvent conventional for such reactions in the art, for example, acetonitrile.

In an example of the present disclosure, in the alkylation reaction, the base is a base conventional for such reactions in the art, for example carbonate or bicarbonate, more preferably carbonate, for example, potassium carbonate.

In an example of the present disclosure, the alkylation is carried out at a temperature conventional for such reactions in the art, for example, 70° C. to 90° C.

In an example of the present disclosure, a progress of the alkylation reaction is detected using a conventional monitoring approach in the art (e.g., TLC, HPLC, or NMR), and typically the time when the compound of Formula (VI) disappears or no longer reacts is taken as a reaction endpoint. A duration of the alkylation reaction may be in the range of 20 hours to 28 hours.

The present disclosure further provides an isotope-labeled compound of formula (II) or a salt thereof:

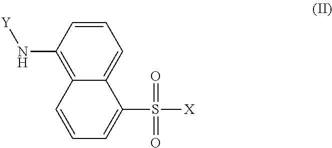

wherein Y is

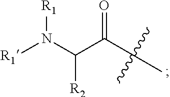

wherein definitions of R₁, R_{1′}, and R₂ are as recited above; and

wherein at least one atom in Y is substituted by a heavier isotope thereof.

In an example of the present disclosure, at least one ¹H in Y is substituted by a heavier isotope ²H thereof.

In an example of the present disclosure, at least one ¹²C in Y is substituted by a heavier isotope ¹³C thereof.

In an example of the present disclosure, at least one ¹⁴N in Y is substituted by a heavier isotope ¹⁵N thereof.

In an example of the present disclosure, at least one ¹⁶O in Y is substituted by a heavier isotope ¹⁸O thereof.

In an example of the present disclosure, the isotope-labeled compound of formula (II) is any one of the following compounds:

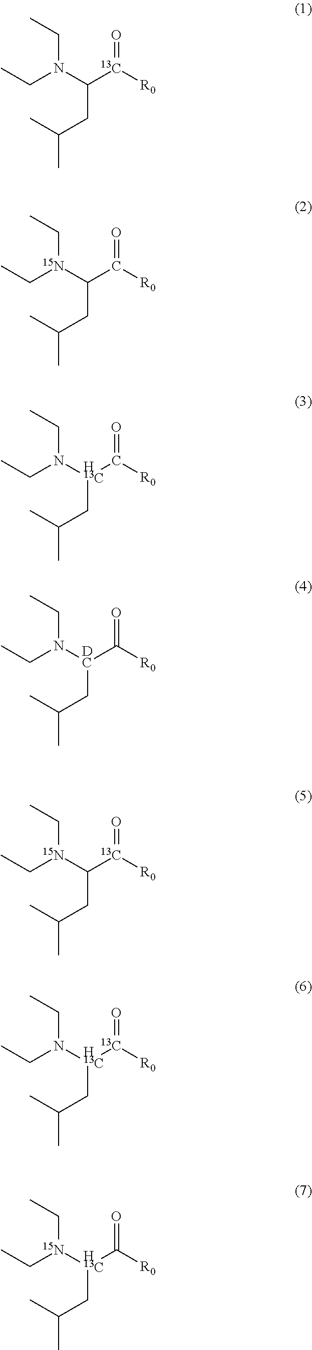

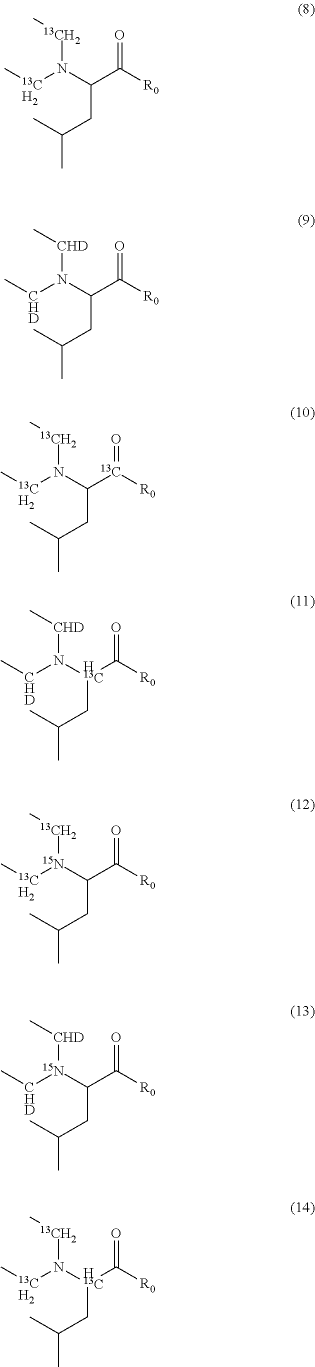

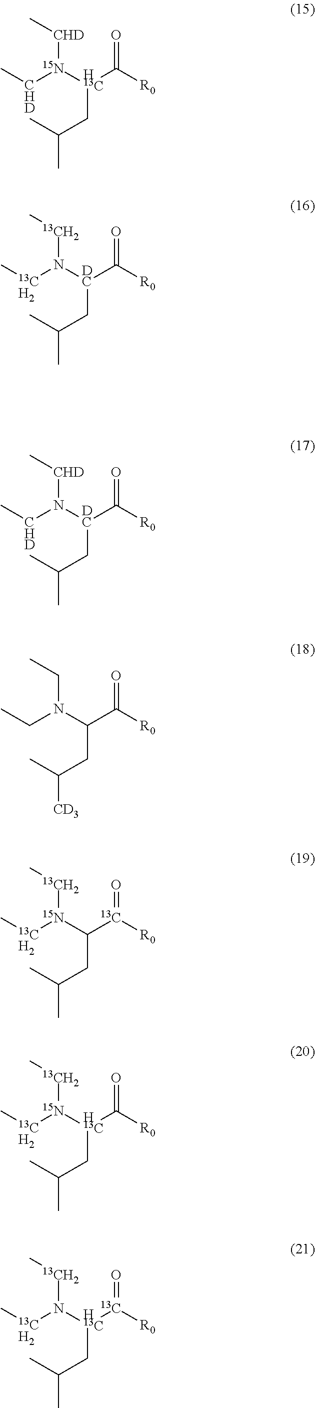

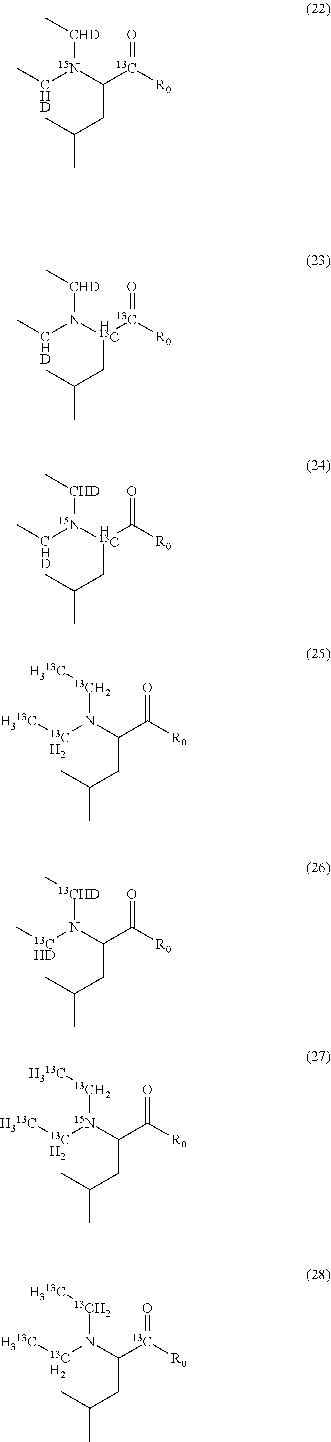

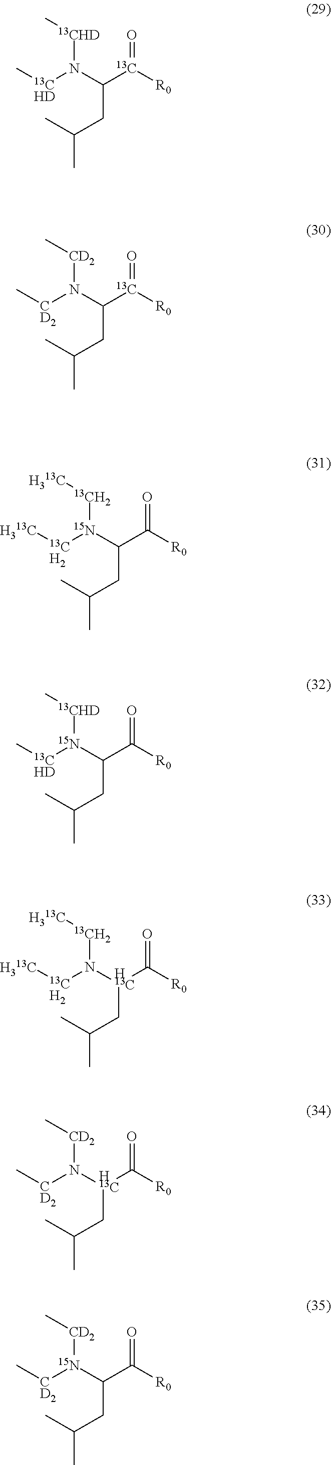

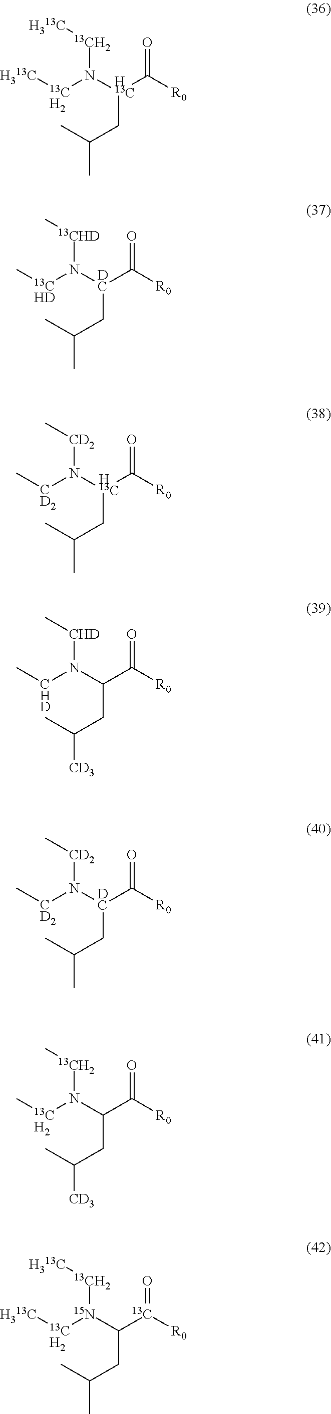

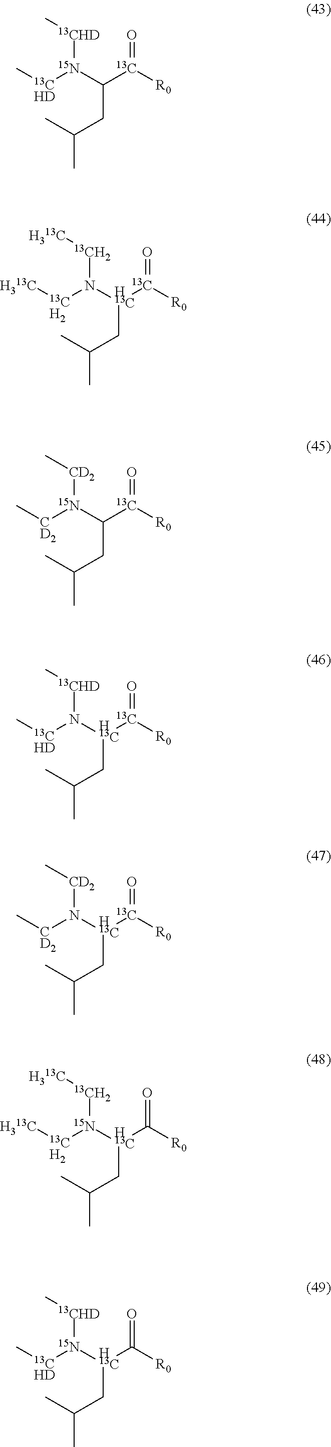

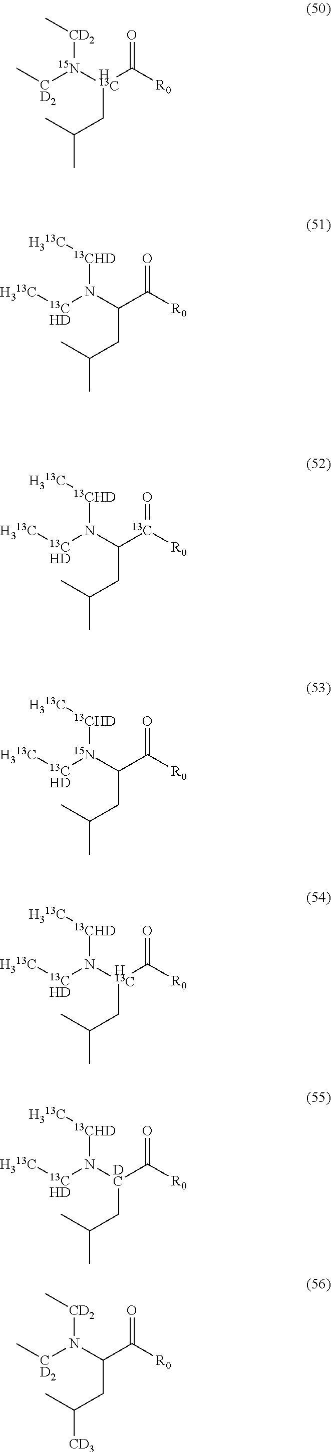

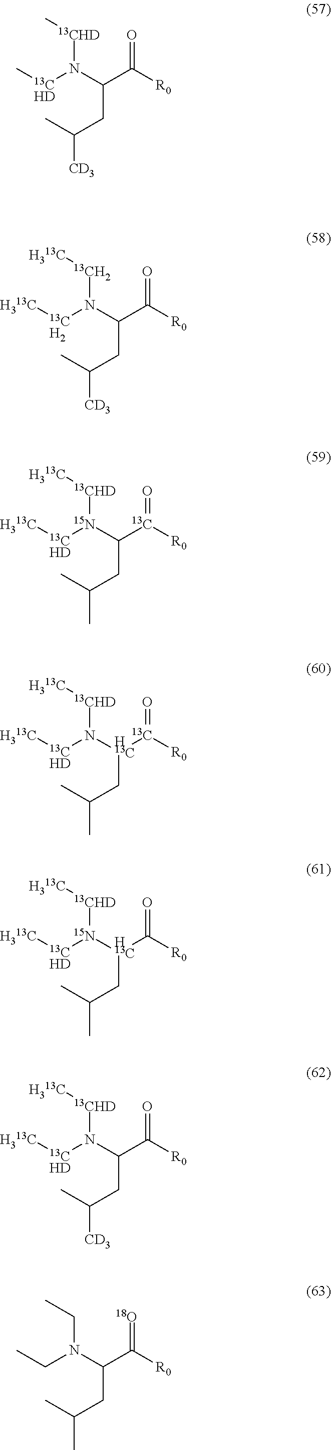

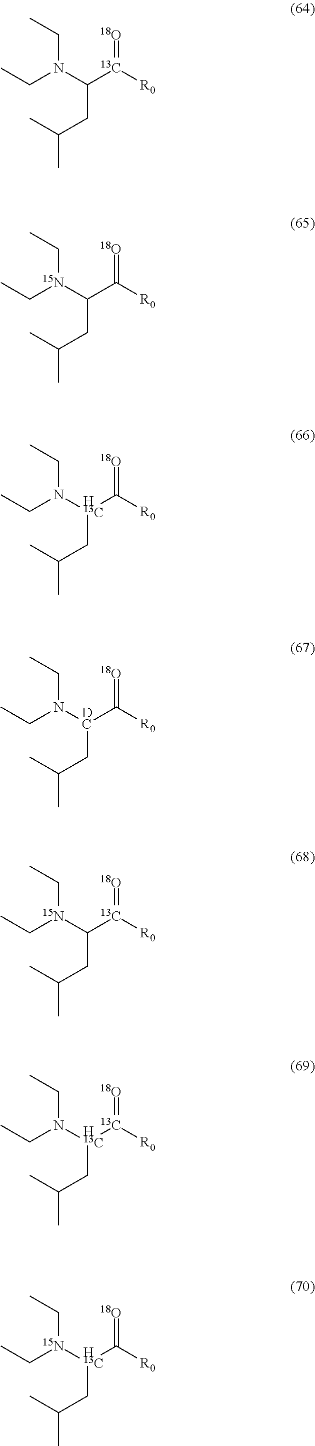

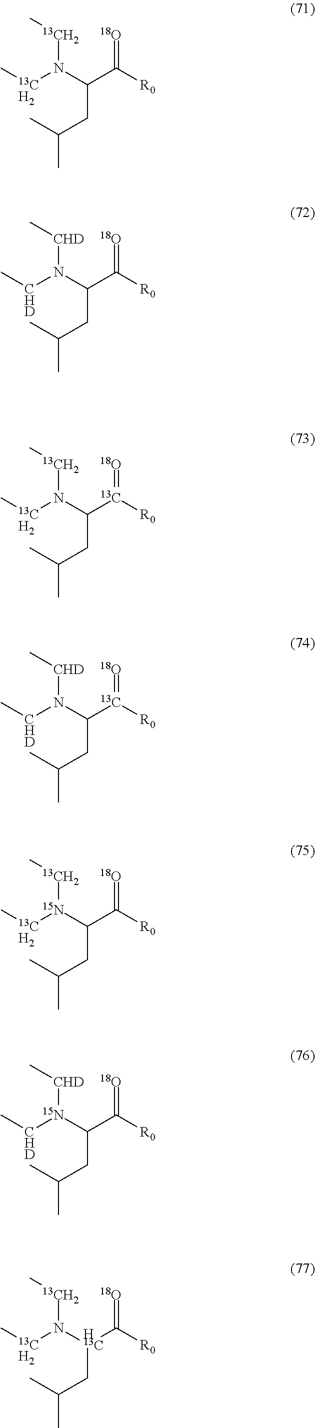

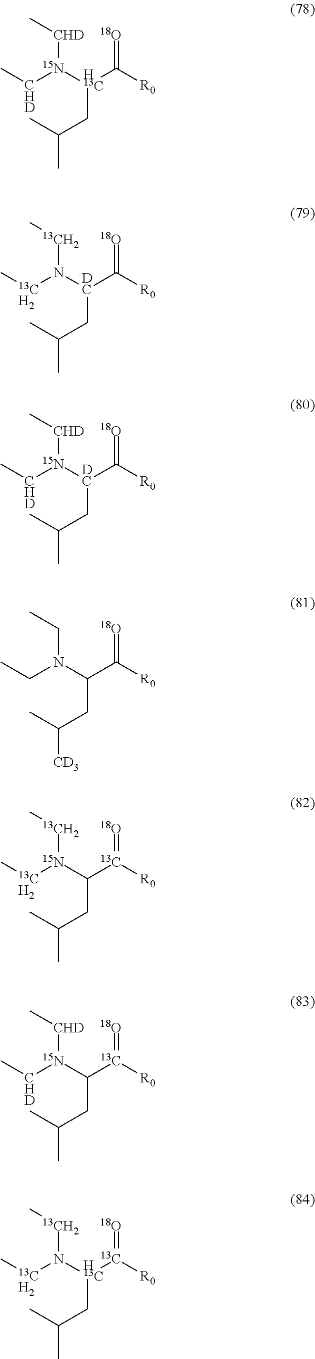

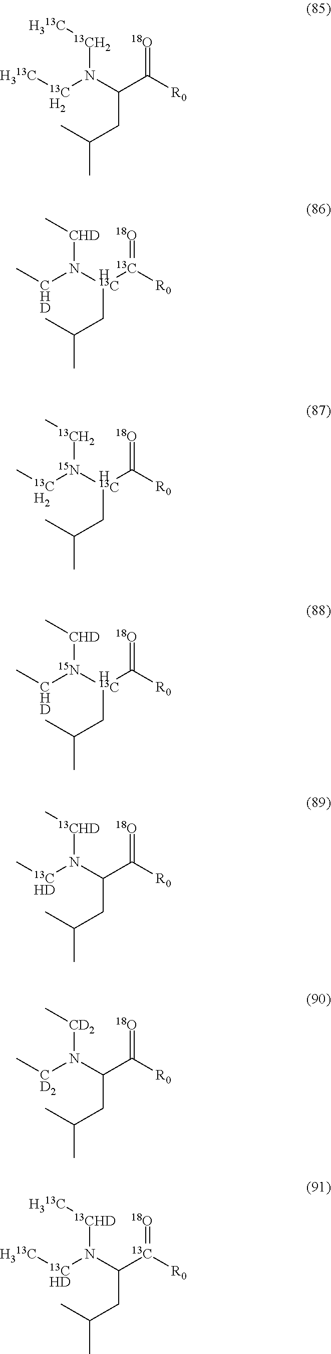

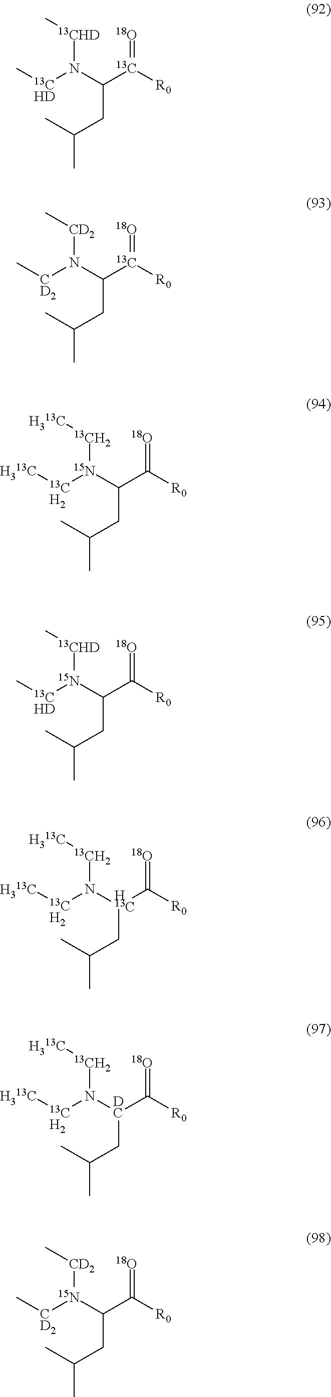

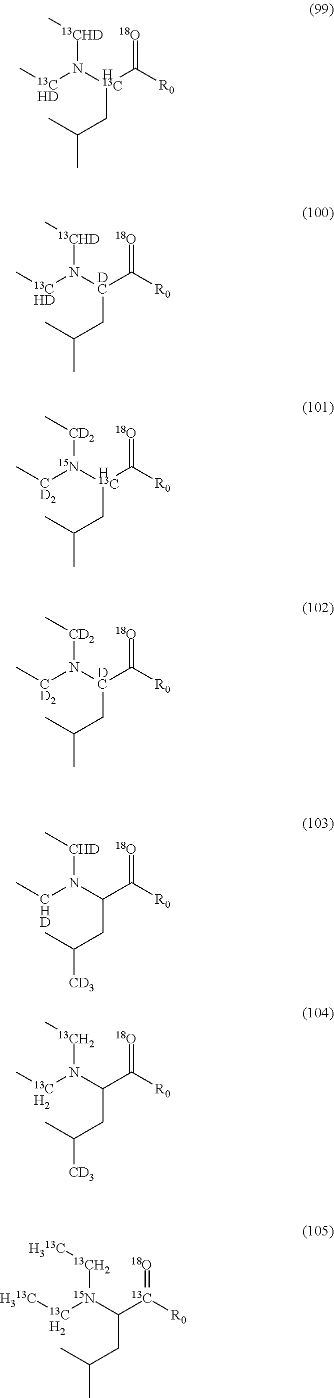

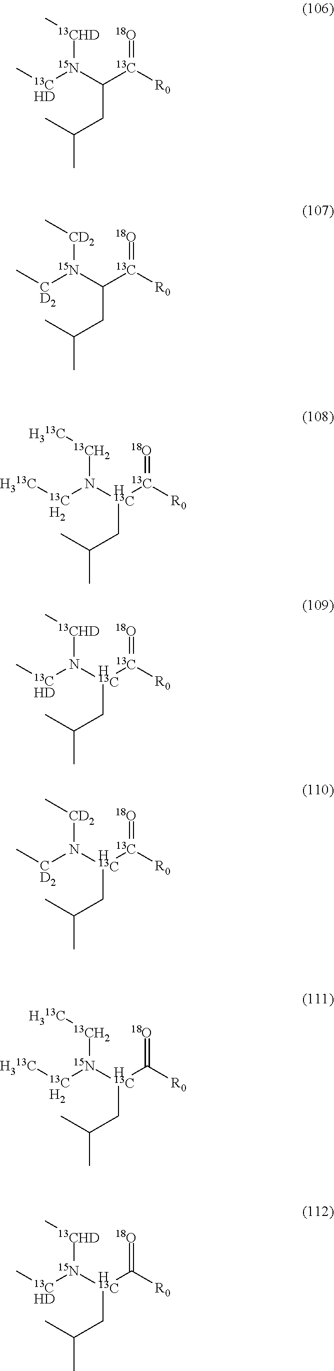

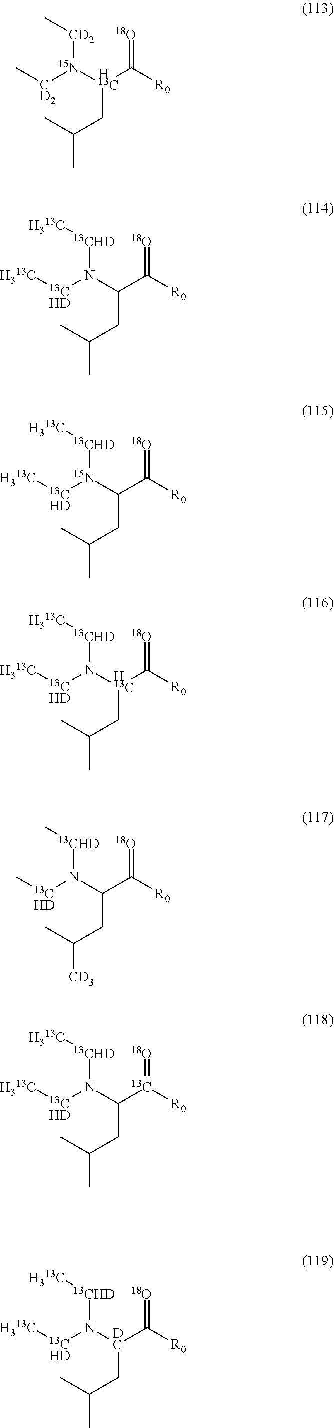

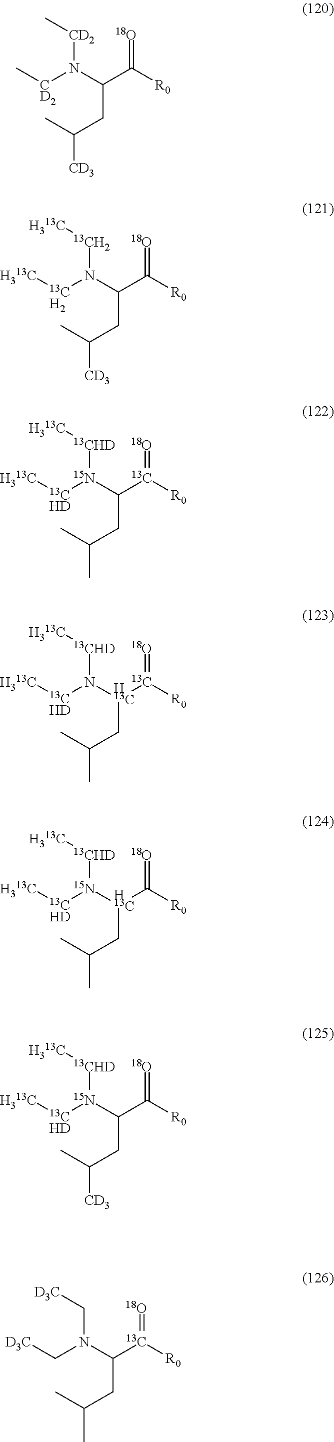

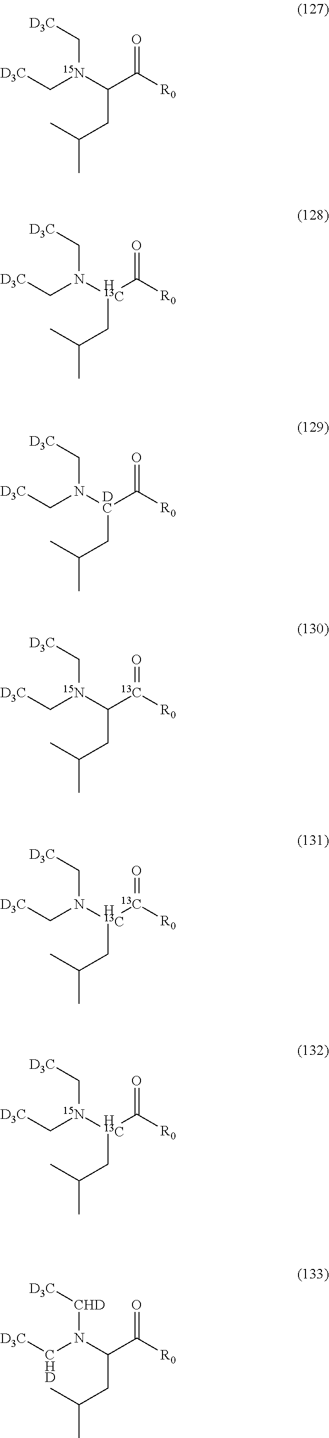

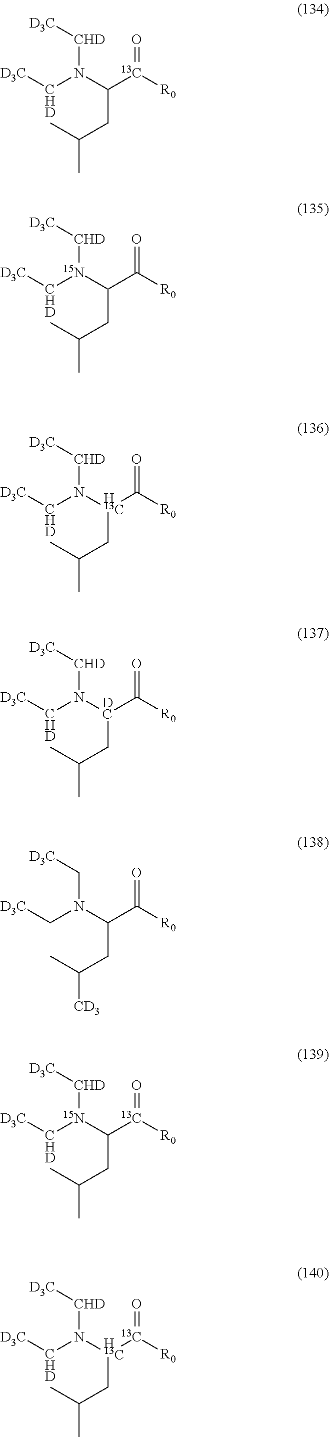

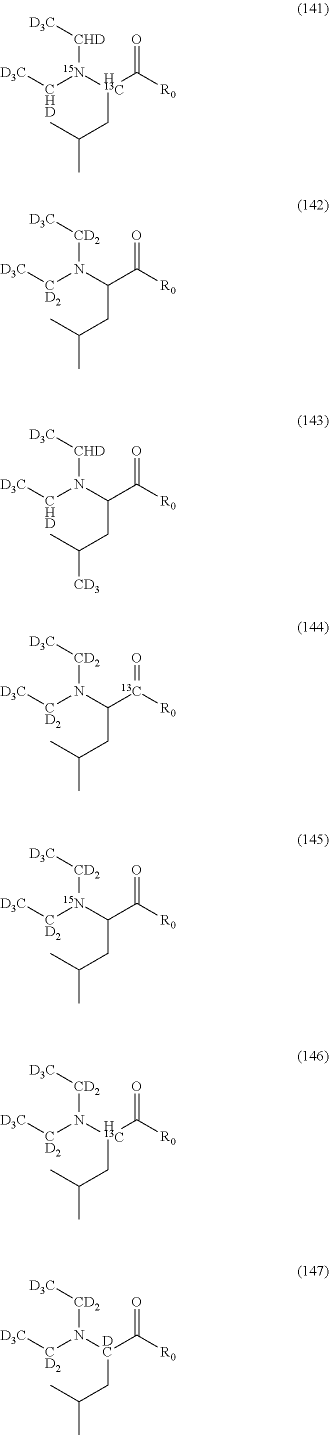

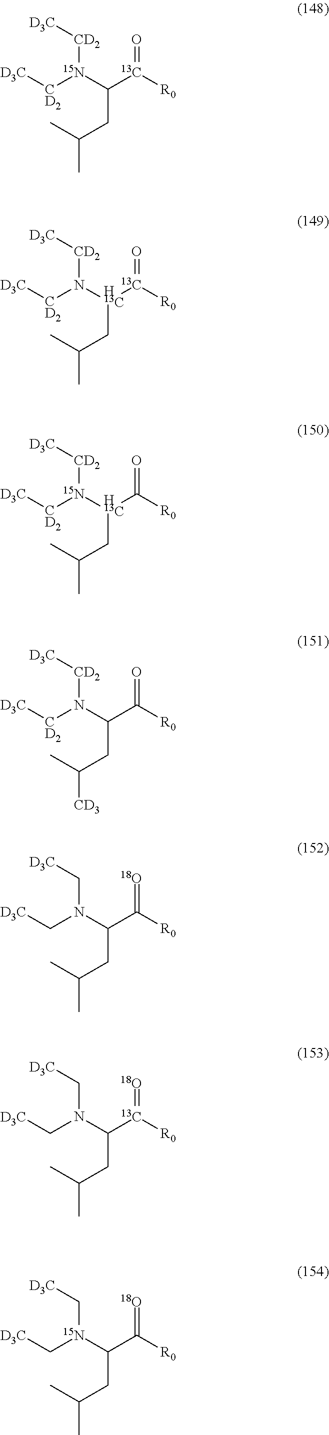

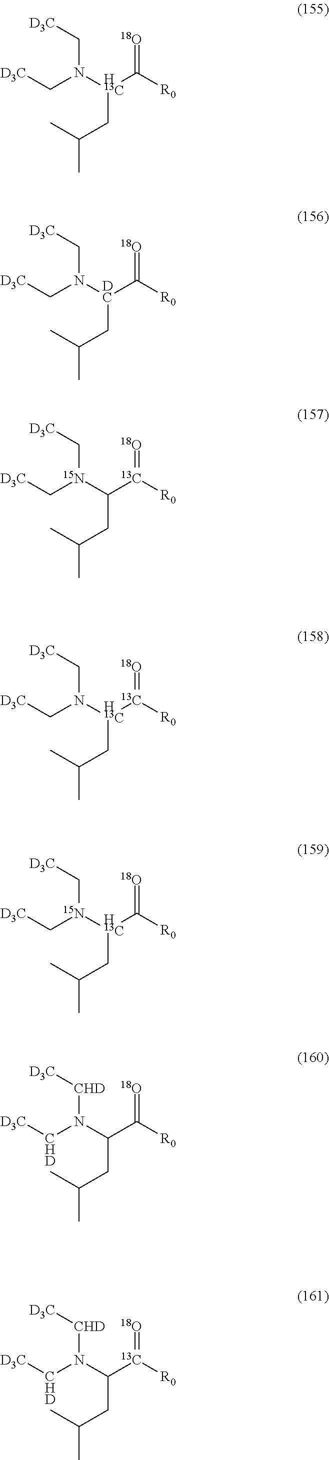

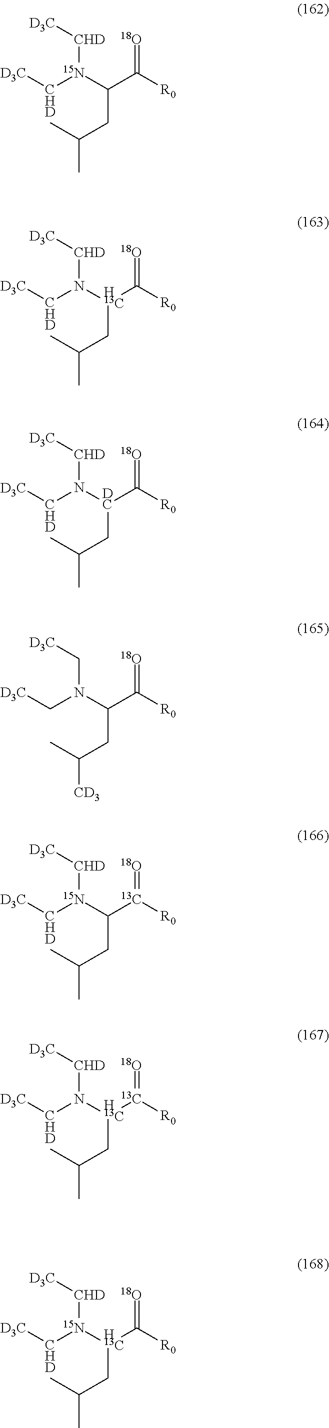

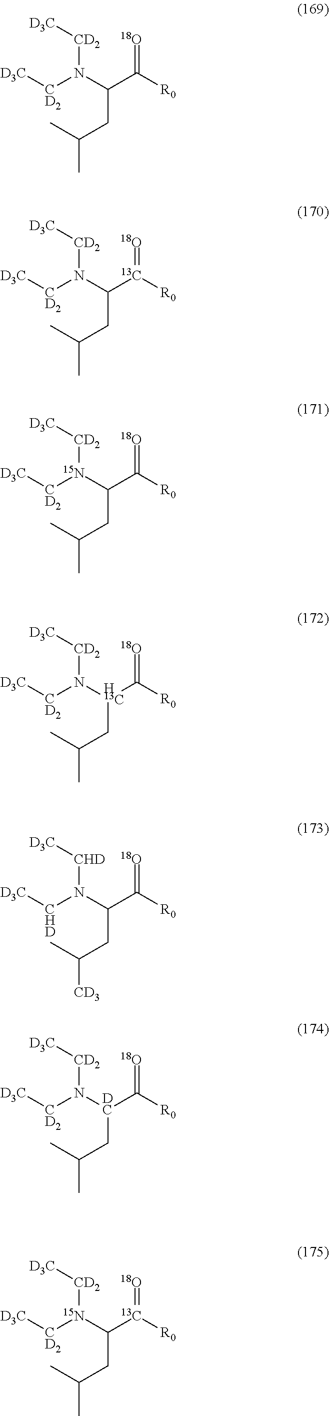

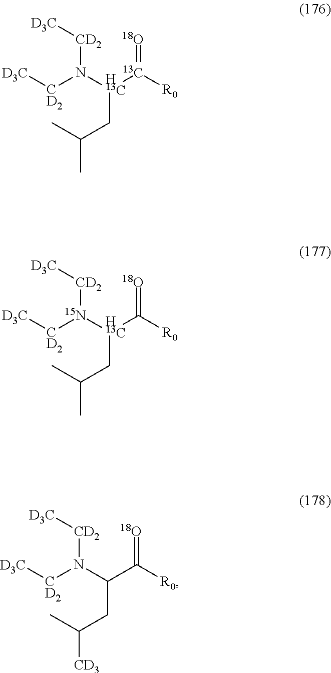

wherein R₀ is

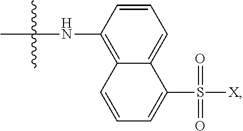

### and X is OH or Cl.

The isotope-labeled compound of formula (II) or the salt thereof may be prepared by conventional methods in the art, for example, ²H, ¹³C, ¹⁵N-labeled isotope-labeled compounds are prepared by the corresponding commercially available isotope-labeled acetaldehyde and isotope-labeled sodium cyanoborohydride, and ¹⁸O-labeled isotope-labeled compounds are prepared by ¹⁶O—¹⁸O oxygen exchange reaction alone.

The present disclosure further provides an application of the compound of formula (I) or the salt thereof as described above, the isotope-labeled compound of formula (II) or the salt thereof as described above as a derivatization reagent for detecting and/or separating compounds containing hydroxyl and/or amino groups, wherein the compounds containing hydroxyl and/or amino groups further include nucleoside metabolites.

Unless otherwise specified, the terms used herein have the following meanings:

It is to be understood by those skilled in the art that, in accordance with the conventions used in the art, the use of “ [symbol] ” in the formulas describing groups herein means that the corresponding group is attached to other moieties, groups, in the compound by the site.

The term “halogen” refers to fluorine, chlorine, bromine, or iodine.

The term “alkyl” refers to a straight-chain or branched-chain alkyl group having a specific number of carbon atoms. Examples of the alkyl group include methyl, ethyl, n-propyl, isopropyl, n-butyl, tert-butyl, isobutyl, sec-butyl, n-pentyl, n-hexyl, n-heptyl, and similar alkyl groups.

The above preferred conditions may be randomly combined based on the common knowledge in the art, and thus various preferred embodiments of the present disclosure may be derived.

The reagents and starting materials used in the present disclosure are commercially available.

The significant progressive effect of the present disclosure lies in: The present disclosure provides several types of compounds with N,N-dialkylaminoethylaminonaphthoyl and sulfonylation derivatives thereof, as well as synthetic methods thereof. These compounds serve as a specific derivatization reagent capable of reacting with hydroxyl and amino groups, exhibit high reactivity, are cost-effective and readily available, and improve the chromatographic separation behaviors of target compounds and enhance the detection sensitivity of these compounds.

### DETAILED DESCRIPTION

Hereinafter the present invention is further described with reference to the embodiments. However, the present disclosure is not limited to the scope as defined by the embodiments described. Experimental methods with specific conditions unstated in the following embodiments are routine methods with customary conditions that are readily known by a person skilled in the art, or selected in accordance with the specifications of relevant products.

The sources of experimental reagents in the following examples are as listed in Table 1.

TABLE 1
Experimental reagents and sources

| Reagent | Source |
|---|---|
| L-leucine | Aladdin |
| Acetaldehyde | Aladdin |
| 4-(4,6-dimethoxytriazin-2-yl)-4- | Aladdin |
| methylmorpholine hydrochloride (DMTMM) | |
| N,N-dimethylformamide (DMF) | Aladdin |
| Dichloromethane | Aladdin |
| Tetrahydrofuran (THF) | Aladdin |
| Methanol | Sigma |
| Acetonitrile (ACN) | Sigma |
| Deuterated methanol (MeOD) | Sigma |
| Deuterated acetonitrile (CD₃CN) | Sigma |
| Deuterium oxide (D₂O) | Sigma |
| 4-methylmorpholine (NMM) | Sigma |
| 5-aminonaphthalene sulfonic acid (ANSA) | TCI |
| Sodium bicarbonate, petroleum ether, ethyl | China National Pharmaceutical Group |
| acetate | |
| Double-distilled water | Prepared by a double-distillation apparatus |
| | using purified water |
| Reverse-phase filler | DAISOCEL SP-120-50-ODS-RPC |

Qualitative analysis of each starting material and product was performed by an AB Sciex ExionLC UHPLC system, which included a PDA detector, an auto-sampler, a binary gradient pump, a temperature control unit, and the like modules, and was equipped with an ACQUITY UPLC HSS T3 C18 reverse-phase chromatographic column (1.8 am, 2.1 mm×100 mm). Experiments such as molecular weight and mass spectrometric cleavage of each starting material and product were performed on an AB Sciex X 500R TOE. Fine purification of N,N-diethyl leucyl amido naphthalene sulfonic acid was achieved by an Agilent 1100 series LC system, which included a VWD detector, an auto-sampler, a binary gradient pump, and a temperature control unit, and was equipped with a YMC Pack GDS-A C18 chromatographic was column (5 am, 10 mm×25 mm). Structure and purity information for the starting materials and products in synthetic steps was provided by Bruker Ascend 600 MHz NMR. Anke N-1001D-OSB2100 rotary evaporator was used to remove organic solvent, Christ ALPHA 1-2 LD plus freeze dryer was used to remove water, and glass instruments (Beijing Xinweier Instrument Co.) such as the chromatography column were used to complete the synthesis reaction at each step.

### Example 1 Synthesis of N,N-diethyl-L-leucine

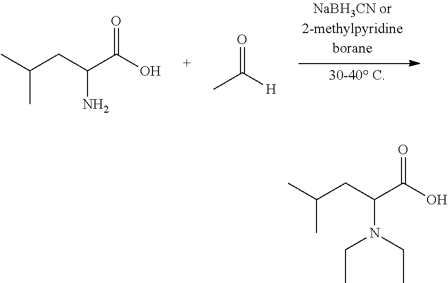

L-leucine powder (800 mg, 6 mmol) was first weighed into a 100 mL round-bottomed flask, 40 mL of sodium acetate or phosphate buffer (0.2 M, pH=2-12) was added and then stirred at 37° C. to dissolve, then sodium cyanoborohydride powder (1.6 g, 24 mmol) was added, and then a acetaldehyde solution (3.4 mL, 60 mmol) was dropwise added. The reaction mixture was stirred at 30-40° C. for 20-28 hours, and finally a 6 mol/L HCl solution (4 mL, 24 mmol) was added and stirred for 10 min to stop the reaction. The organic reagent was removed using a rotary evaporator, and the reactant was lyophilized using a lyophilizer, and then purified by reverse-phase column chromatography to obtain pure N,N-diethylleucine.

The structure of the pure N,N-diethyl L-leucine was confirmed using NMR and mass spectrometry.

¹H NMR (600 MHz, D₂O buffer, pH 7.4): δ 0.971 (dd, 6H), 1.298 (t, 6H), 1.650 (m, 2H), 1.760 (m, 1H), 3.247 (m, 4H), 3.668 (dd, 1H); MS+(TOF) m/z 188.1645.

### Example 2 Synthesis of N,N-diethyl-L-leucine

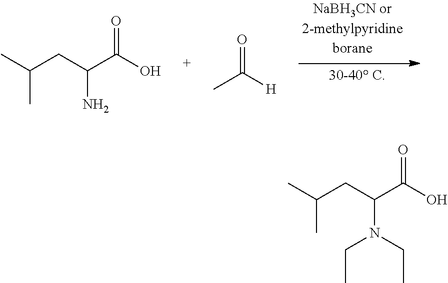

L-leucine powder (800 mg, 6 mmol) was first weighed into a 100 mL round-bottomed flask, 40 mL of sodium acetate or phosphate buffer (0.2 M, pH=2-12) was added and then stirred at 37° C. to dissolve, then 2-methylpyridine borane (1.3 g, 12 mmol) was added, and then a acetaldehyde solution (3.4 mL, 60 mmol) was dropwise added. The reaction mixture was stirred at 30-40° C. for 20-28 hours, and finally a 6 mol/L HCl solution (4 mL, 24 mmol) was added and stirred for 10 min to stop the reaction. The organic reagent was removed using a rotary evaporator, the reactant was lyophilized using a lyophilizer, and then purified by reverse-phase column chromatography to obtain pure N,N-diethylleucine.

### Example 3 Synthesis of N,N-diethyl-L-leucine

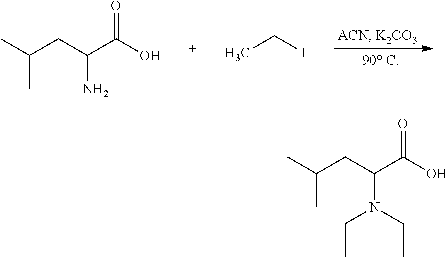

Leucine powder (800 mg, 6 mmol) was first weighed into a 100 mL round-bottomed flask, ground potassium carbonate powder (4.8 g, 6 mmol) and 40 mL of acetonitrile were added, and an iodoethane solution (9.6 mL, 120 mmol) was dropwise added under stirring. The reaction mixture was reacted under reflux at 90° C. for 20-28 hours. Excess potassium carbonate was removed by filtration and the solvent was evaporated. The crude product is filtered by addition of diethyl ether, and the precipitate was washed several times with diethyl ether and finally recrystallized from acetonitrile to obtain a purified product.

### Example 4 Synthesis of N,N-diethyl leucylamino naphthalene sulfonic acid

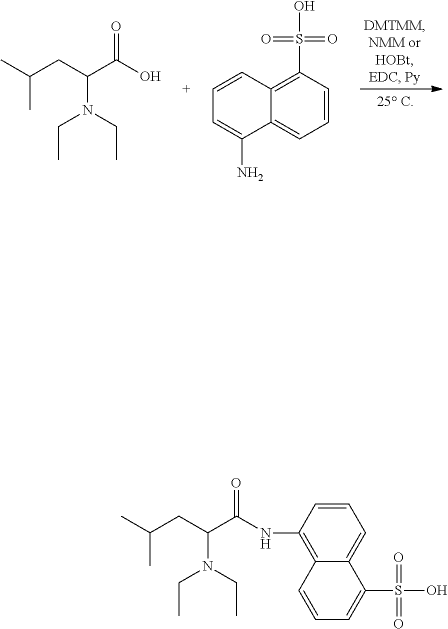 [SMILES: CCN(CC)C(CC(C)C)C(=O)O ; Nc1cccc2c(S(=O)(=O)O)cccc12 ; CCN(CC)C(CC(C)C)C(=O)Nc1cccc2c(S(=O)(=O)O)cccc12]

N,N-diethylleucine (800 μL, 16 mmol) was dissolved in DMF, DMTMM (6.9 mg, 24 mmol) was added, NMM (43.3 μL, 320 mmol) was dropwise added and vortexed for a moment, and 5-aminonaphthalene sulfonic acid powder (71.6 mg, 640 mmol) was added but not vortex. The reaction mixture was gently placed on a metal shaker, reacted for 8-24 hours at room temperature in 12 groups. The product was purified by extraction, and 192 mL of dichloromethane and 19.2 mL of double distilled water were added to extract impurities to obtain a supernatant.

### Example 5 Synthesis of N,N-diethyl leucylamino naphthalene sulfonic acid

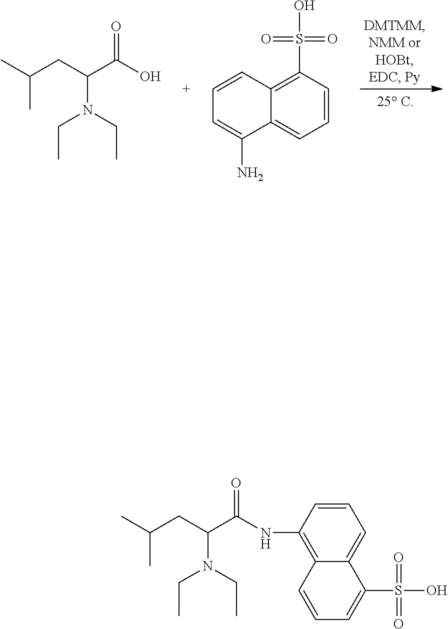 [SMILES: CCN(CC)C(CC(C)C)C(=O)O ; Nc1cccc2c(S(=O)(=O)O)cccc12 ; CCN(CC)C(CC(C)C)C(=O)Nc1cccc2c(S(=O)(=O)O)cccc12]

N,N-diethylleucine (35.7 mg, 192 mmol) was dissolved in DMF and activated by the addition of 1.2 equivalents of EDC (44.2 mg, 230 mmol) and HOBt (31.1 mg, 230 mmol). 1.5 equivalents of 5-aminonaphthalene sulfonic acid (64.4 mg, 287.5 mmol) were added thereto, and 2 mL of pyridine was dropwise added thereto, and the reaction was allowed to proceed at normal temperature overnight with stirring.

The organic reagent was removed using a rotary evaporator and the crude product was purified by reverse-phase column chromatography using a pad of ODS C18 to obtain about 26 mg of yellow-brown powder. Fine purification was carried out using semi-preparative liquid chromatography Agilent 1100 LC-VWD coupled to a rotary evaporator to remove the solvent to obtain a pure product.

The structure of the pure product was confirmed using NMR and mass spectrometry.

¹H NMR (600 MHz, meOD): δ 1.232, 1.070 (dd, 6H), 1.435 (t, 6H), 1.832 (m, 2H), 2.050 (m, 1H), 3.411, 3.502 (q, 4H), 4.359 (dd, 1H), 7.213 (m, 1H), 7.402 (m, 1H), 7.457 (m, 1H), 7.692 (m, 1H), 8.037 (m, 1H), 8.722 (m, 1H); MS+(TOF) m/z 393.1845.

### Example 6 Synthesis of N,N-diethyl leucyl amido naphthalene sulfonyl chloride

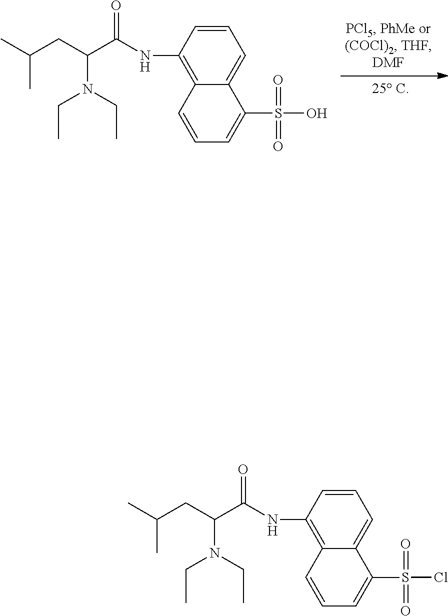 [SMILES: CCN(CC)C(CC(C)C)C(=O)Nc1cccc2c(S(=O)(=O)O)cccc12 ; CCN(CC)C(CC(C)C)C(=O)Nc1cccc2c(S(=O)(=O)Cl)cccc12]

N,N-diethyl leucylaminonaphthalenesulfonic acid (26.1 mg, 0.07 mmol) was weighed and dissolved in 5 mL of toluene as a solvent under sonication at a molar ratio of 1:50, and excess phosphorus pentachloride (0.7 g, 3.33 mmol) was weighed into a reaction flask. The reaction mixture was allowed to react at room temperature for 1-3 hours. Ice ethyl acetate was added for extraction, an ice saturated sodium bicarbonate solution was gradually dropwise added to quench the reaction, pH was adjusted to 7, and the supernatant is taken and evaporated to dryness to obtain an N,N-diethyl leucyl amido naphthalene sulfonyl chloride crude product.

The crude product was purified using normal phase column chromatography packed with silica gel, petroleum ether, ethyl acetate, and acetonitrile as eluents, 1:1 (acetonitrile/ethyl acetate) and 1:2 (acetonitrile/ethyl acetate) elution fractions were combined and the solvent was dried up by rotary evaporation to obtain pure N,N-diethyl leucyl amido naphthalenesulfonyl chloride.

The structure of the pure product was confirmed using NMR.

¹H NMR (600 MHz, CD₃CN): δ 1.203 (dd, 6H), 1.558 (t, 6H), 1.958 (m, 2H), 7.892 (m, 1H), 8.026 (m, 1H), 8.135 (m, 1H), 8.593 (m, 1H), 8.816 (m, 1H), 8.964 (m, 1H); MS+(TOF) m/z 411.1495.

### Example 7 Synthesis of N,N-diethyl leucyl amido naphthalene sulfonyl chloride (DELANS-Cl)

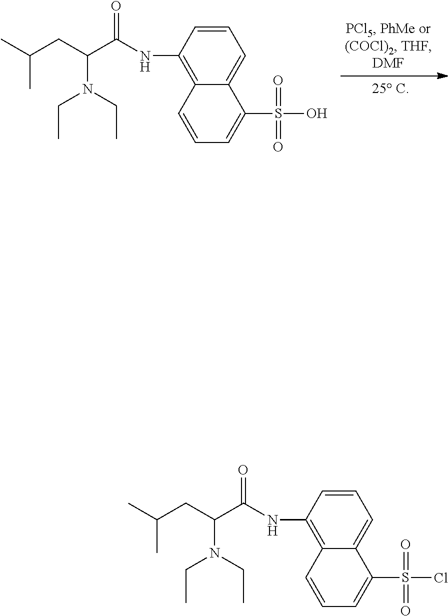 [SMILES: CCN(CC)C(CC(C)C)C(=O)Nc1cccc2c(S(=O)(=O)O)cccc12 ; CCN(CC)C(CC(C)C)C(=O)Nc1cccc2c(S(=O)(=O)Cl)cccc12]

N,N-diethyl leucylaminonaphthalenesulfonic acid (26.1 mg, 0.067 mmol) was weighed and dissolved in 4 mL THF. 20 equivalents of oxalyl chloride (112 μL, 1.33 mmol) were diluted to 500 μL of THF in an ice bath and added into a reaction flask. With 3 drops of DMF added, the reaction mixture was stirred at room temperature and reacted for 10 minutes to 30 minutes. The THF and oxalyl chloride were removed by rotary evaporation.

The crude product was purified using normal phase column chromatography packed with silica gel, petroleum ether, ethyl acetate, and acetonitrile as eluents, 1:1 (acetonitrile/ethyl acetate) and 1:2 (acetonitrile/ethyl acetate) elution fractions were combined and the solvent was dried up by rotary evaporation to obtain pure N,N-diethyl leucyl amido naphthalenesulfonyl chloride.

### Example 8 Synthesis of d₂-N,N-diethyl L-leucine

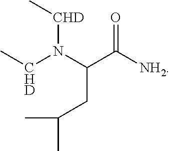

### d₂-N,N-diethyl L-leucine

The synthesis steps for d₂-N,N-diethyl L-leucine were the same as those for d₂-N,N-diethyl L-leucine, except that deuterated sodium cyanoborohydride was used as a starting material for the synthesis. (M+H]⁺=190.1771)

### Example 9 Synthesis of d₂-N,N-diethyl leucylamino naphthalene sulfonic acid (d2-DELANS-Cl)

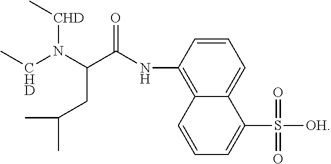

### d₂-N,N-diethyl leucylamino naphthalene sulfonic acid

The synthesis steps for d₂-N,N-diethyl leucyl amido naphthalene sulfonic acid were the same as those for N,N-diethyl leucyl amido naphthalene sulfonic acid, except that d₂-N,N-diethyl L-leucine was used as a starting material for the synthesis. (M+H]⁺=395.1968)

### Example 10 Synthesis of d₂-N,N-diethyl leucyl amido naphthalene sulfonyl chloride

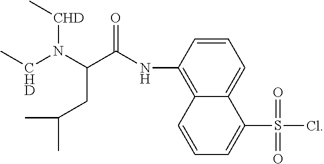

### d₂-N,N-diethyl leucyl amido naphthalene sulfonyl chloride

The synthesis steps for d₂-N,N-diethyl leucyl amido naphthalenesulfonyl chloride were the same as those for N,N-diethyl leucyl amido naphthalenesulfonyl chloride, except that d₂-N,N-diethyl leucylamino naphthalene sulfonic acid was used as a starting material for the synthesis.

The structure of the pure product was confirmed using NMR and mass spectrometry.

¹H NMR (600 MHz, CD₃CN): δ 1.203 (dd, 6H), 1.518 (d, 3H), 1.592 (d, 3H), 1.959 (m, 2H), 3.479 (m, 2H), 4.388 (m, 1H), 7.875 (m, 1H), 8.038 (m, 1H), 8.132 (m, 1H), 8.584 (m, 1H), 8.831 (m, 1H), 8.959 (m, 1H); MS+(TOF) m/z 413.1602.

### Effects Example 1 Derivatization Reaction of the Derivatization Reagent DELANS-Cl on Nucleoside Metabolites

### N,N-diethyl leucyl amido naphthalene sulfonyl chloride (DELANS-Cl

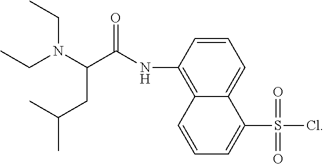 [SMILES: CCN(CC)C(CC(C)C)C(=O)Nc1cccc2c(S(=O)(=O)Cl)cccc12]

### d₂-N,N-diethyl leucylamino naphthalene sulfonic acid (d₂-DELANS-Cl

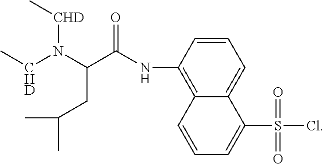

The derivatization reagents described above can be used to derivatize amino-, hydroxyl-containing compounds like the classical dansyl chloride, and can also be used to derivatize nucleosides and are described below.

### Preparation of Working Solution:

Powder of nucleoside metabolites listed in Table 2 was accurately weighed separately, and dissolved in a sodium carbonate/sodium bicarbonate buffer solution with a concentration of 250 mM and pH of 9.4 to obtain a standard stock solution of each metabolite at a concentration listed in Table 2. 50 μL of each of the single standard solutions was mixed to obtain a mixed stock solution of 65 nucleoside metabolites, which was an initial mixed stock solution. The initial mixed stock solution was diluted to obtain a working solution S₁ with a total concentration of about 4 mM, and then the solution was diluted step by step according to a dilution ratio of 1:2:4:10:20:40:100:200:400:1000:2000:4000 to obtain a working solution of totally 12 concentration gradients of S₁, S₂, S₃, S₄, S₅, S₆, S₇, S₈, S₉, S₁₀, S₁₁ and S₁₂.

TABLE 2
Concentrations of substances in standard stock solution, initial mixed stock
solution, working solution S₁, and S₁ derivative reaction solution

| | | | | | Concentration |
|---|---|---|---|---|---|
| | | | | Concentration | of each |
| | | Concentration | Concentration | of each | substance in |
| | | of standard | of initial | substance in | derivative |
| | | stock | mixed stock | working | reaction |
| | | solution | solution | solution S₁ | solution S₁ |
| No. | Metabolite | (mM) | (mM) | (mM) | (mM) |
| 1 | Adenine | 10.00 | 0.15 | 0.0410 | 911.68 |
| 2 | Guanine | 10.00 | 0.15 | 0.0410 | 911.68 |
| 3 | Hypoxanthine | 10.00 | 0.15 | 0.0410 | 911.68 |
| 4 | Xanthine | 10.00 | 0.15 | 0.0410 | 911.68 |
| 5 | Uracil | 15.00 | 0.23 | 0.0615 | 1367.52 |
| 6 | Cytosine | 10.00 | 0.15 | 0.0410 | 911.68 |
| 7 | Thymine | 10.00 | 0.15 | 0.0410 | 911.68 |
| 8 | Adenosine | 10.00 | 0.15 | 0.0410 | 911.68 |
| 9 | Guanosine | 6.91 | 0.11 | 0.0284 | 630.15 |
| 10 | Uridine | 50.00 | 0.77 | 0.2051 | 4558.40 |
| 11 | Cytidine | 10.00 | 0.15 | 0.0410 | 911.68 |
| 12 | Thymidine | 10.00 | 0.15 | 0.0410 | 911.68 |
| 13 | Inosine | 10.00 | 0.15 | 0.0410 | 911.68 |
| 14 | Xanthosine | 10.00 | 0.15 | 0.0410 | 911.68 |
| 15 | Deoxyadenosine | 10.00 | 0.15 | 0.0410 | 911.68 |
| 16 | Deoxyguanosine | 10.00 | 0.15 | 0.0410 | 911.68 |
| 17 | Deoxyuridine | 10.00 | 0.15 | 0.0410 | 911.68 |
| 18 | Deoxycytidine | 10.00 | 0.15 | 0.0410 | 911.68 |
| 19 | Deoxyinosine | 10.00 | 0.15 | 0.0410 | 911.68 |
| 20 | 1-methyladenosine | 10.00 | 0.15 | 0.0410 | 911.68 |
| 21 | 2-methyladenosine | 10.00 | 0.15 | 0.0410 | 911.68 |
| 22 | N⁶-methyladenosine | 10.00 | 0.15 | 0.0410 | 911.68 |
| 23 | 2′-O-methyladenosine | 15.00 | 0.23 | 0.0615 | 1367.52 |
| 24 | N⁶,2′-O-dimethyladenosine | 10.00 | 0.15 | 0.0410 | 911.68 |
| 25 | N¹,2′-O-dimethyladenosine | 10.00 | 0.15 | 0.0410 | 911.68 |
| 26 | 3′-O-methyladenosine | 10.00 | 0.15 | 0.0410 | 911.68 |
| 27 | 8-methyladenosine | 10.00 | 0.15 | 0.0410 | 911.68 |
| 28 | 3-methylcytidine | 10.00 | 0.15 | 0.0410 | 911.68 |
| 29 | N⁴-methylcytidine | 10.00 | 0.15 | 0.0410 | 911.68 |
| 30 | 5-methylcytidine | 10.00 | 0.15 | 0.0410 | 911.68 |
| 31 | 5-hydroxymethylcytidine | 10.00 | 0.15 | 0.0410 | 911.68 |
| 32 | 2′-O-methylcytidine | 50.00 | 0.77 | 0.2051 | 4558.40 |
| 33 | N⁴,2′-O-dimethylcytidine | 10.00 | 0.15 | 0.0410 | 911.68 |
| 34 | 2′-C-methylcytidine | 10.00 | 0.15 | 0.0410 | 911.68 |
| 35 | 5-methyl-2′-O-methylcytidine | 50.00 | 0.77 | 0.2051 | 4558.40 |
| 36 | 1-methylguanosine | 10.00 | 0.15 | 0.0410 | 911.68 |
| 37 | 2-methylguanosine | 4.00 | 0.06 | 0.0164 | 364.67 |
| 38 | N²,N²-dimethylguanosine | 10.00 | 0.15 | 0.0410 | 911.68 |
| 39 | 7-methylguanosine | 10.00 | 0.15 | 0.0410 | 911.68 |
| 40 | 2′-O-methylguanosine | 10.00 | 0.15 | 0.0410 | 911.68 |
| 41 | 5-methyluridine | 10.00 | 0.15 | 0.0410 | 911.68 |
| 42 | 2′-O-methyluridine | 10.00 | 0.15 | 0.0410 | 911.68 |
| 43 | 5-methyl-2′-O-methyluridine | 10.00 | 0.15 | 0.0410 | 911.68 |
| 44 | 2′-O-methoxyinosine | 10.00 | 0.15 | 0.0410 | 911.68 |
| 45 | 5-methyldeoxycytidine | 10.00 | 0.15 | 0.0410 | 911.68 |
| 46 | 5-hydroxymethyldeoxycytidine | 50.00 | 0.77 | 0.2051 | 4558.40 |
| 47 | N⁶-methyldeoxyadenosine | 100.00 | 1.54 | 0.4103 | 9116.81 |
| 48 | 3-methyladenine | 11.05 | 0.17 | 0.0453 | 1007.41 |
| 49 | 5′-methylcytosine | 10.00 | 0.15 | 0.0410 | 911.68 |
| 50 | 5-hydroxymethylcytosine | 15.00 | 0.23 | 0.0615 | 1367.52 |
| 51 | 5-formylcytosine | 10.00 | 0.15 | 0.0410 | 911.68 |
| 52 | 5-carboxylcytosine | 15.00 | 0.23 | 0.0615 | 1367.52 |
| 53 | 7-methylguanine | 50.00 | 0.77 | 0.2051 | 4558.40 |
| 54 | 5,6-dihydrouracil | 10.00 | 0.15 | 0.0410 | 911.68 |
| 55 | 2-aminoadenosine | 10.00 | 0.15 | 0.0410 | 911.68 |
| 56 | 8-aminoadenosine | 10.00 | 0.15 | 0.0410 | 911.68 |
| 57 | 8-oxoadenosine | 10.00 | 0.15 | 0.0410 | 911.68 |
| 58 | Dihydrouridine | 10.00 | 0.15 | 0.0410 | 911.68 |
| 59 | Pseudouridine | 10.00 | 0.15 | 0.0410 | 911.68 |
| 60 | Orotidine | 10.00 | 0.15 | 0.0410 | 911.68 |
| 61 | 5-methoxycarbonylmethyluridine | 10.00 | 0.15 | 0.0410 | 911.68 |
| 62 | 5-aminomethylcarbamoylmethyluridine | 4.00 | 0.06 | 0.0164 | 364.67 |
| 63 | 5-methoxycarbonylmethyl-2- | 10.00 | 0.15 | 0.0410 | 911.68 |
| | thiouridine | | | | |
| 64 | 5-methoxycarbonylmethyl-2′-O- | 10.00 | 0.15 | 0.0410 | 911.68 |
| | methyluridine | | | | |
| 65 | N⁴-acetylcytidine | 8.40 | 0.13 | 0.0345 | 765.81 |

### Preparation of Internal Standard Solution:

A d₂-DELANS-Cl solution with a concentration of 5 mmol/L was prepared with the solvent of the isotope-labeled derivatization reagent d₂-DELANS-Cl as a dry acetonitrile solution. 50 μL of a working solution S₂ was transferred into a 500 μL EP tube using a pipette, 400 μL of a d2-DELANS-Cl solution (5 mM, dissolved in acetonitrile) was sucked and added thereto, the reaction EP tube was placed on a metal shaker, the reaction temperature was set to 37° C., and the reaction was carried out at a shaking frequency of 900 rpm for 5 hours. The reaction mixture was immediately cooled on ice to quench the reaction. Upon completion of the derivatization reaction, 100 μL of the reaction solution was taken out, diluted 5-fold with an acetonitrile solution, and mixed well to obtain the internal standard solution. The obtained solution was sealed and stored at low temperature of −20° C. or −80° C.

Derivatization Reaction of Derivatization Reagent DELANS-Cl with Standard Solution and Establishment of Linear Curve:

A DELANS-Cl solution with a concentration of 5 mmol/L was prepared with a dry acetonitrile solution as the solvent of the derivatization reagent. First, 5 μL of a standard solution (namely, working solution with various concentration gradients) (dissolved in a sodium bicarbonate buffer solution with a concentration of 250 mM and pH of 9.4) was transferred using a pipette, and placed into a 500 μL EP tube, then 40 μL of a DELANS-Cl solution (5 mM, acetonitrile solution) was sucked and added thereto, the reaction EP tube was placed into a metal shaker, the reaction temperature was set to 37° C., and the reaction was carried out at a shaking frequency of 900 rpm for 5 hours. The reaction mixture solution was then immediately cooled on ice to quench the reaction. Upon completion of the derivatization reaction, 8 μL of the reaction solution was taken out, and diluted 5-fold with the acetonitrile solution, and then 10 μL of an internal standard solution (volume ratio: 4:1) was added and mixed well. Treated samples were sealed and stored at low temperature of −20° C. or −80° C. before being subjected to analysis by the UHPLC-MS system.

Derivatization Reaction of Derivatization Reagent DELANS-Cl with Sample:

The derivatization reaction of nucleoside metabolites in the sample was as follows. 5 L of a sample solution (urine sample, serum sample, tissue sample, and lung cancer cell sample respectively) was taken out into a 1.5 mL EP tube, 40 μL of a DELANS-Cl solution was transferred (5 mM, dissolved in an acetonitrile solution) thereto, the reaction EP tube was placed on a metal shaker, the reaction temperature is set to 37° C., and the reaction was carried out at a shaking frequency of 900 rpm for 5 hours. The reaction mixture solution was immediately cooled on ice to quench the reaction. Finally, upon completion of the derivatization reaction, 8 μL of the reaction solution was taken out, diluted 5-fold with the acetonitrile solution, and 10 μL of internal standard solution (volume ratio: 4:1) was added and mixed well. UHPLC-MS system analysis was prepared.

Urine samples were collected from adult male morning urine. Serum samples were collected from healthy adults in accordance with the relevant requirements of scientific research ethics of Fudan University and national laws. Tissue samples were taken from rabbit liver. Lung cancer cell samples: the cell sample selected in the experiment was non-small cell lung adenocarcinoma cell line A549.

For the derivatization reaction of nucleoside metabolites with derivatization reagents N,N-dimethylamino naphthalene sulfonyl chloride (DNS-Cl) and N,N-diethylamino naphthalene sulfonyl chloride (DENS-Cl), reference is made to DELANS-Cl.

### N,N-dimethylamino naphthalene sulfonyl chloride (DNS-Cl

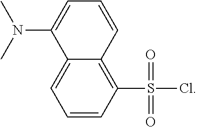 [SMILES: CN(C)c1cccc2c(S(=O)(=O)Cl)cccc12]

### N,N-diethylamino naphthalene sulfonyl chloride (DENS-Cl

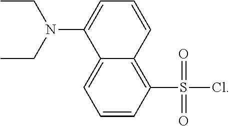 [SMILES: CCN(CC)c1cccc2c(S(=O)(=O)Cl)cccc12]

### Test Method:

The liquid phase was equipped with a Waters ACQUITY UPLC HSST3 C18 reverse-phase chromatographic column (Waters, Technologies, Milford. USA). The column temperature was 40° C., and the autosampler temperature was 4° C. Mobile phase A was 0.1% formic acid in water (MilliQ ultrapure water) and mobile phase B was 0.1% formic acid in acetonitrile. The elution gradient (B %) was as follows: 0-0.5 min: 2-25%, 0.5-3. 6 min: 25%, 3.6-3.7 min: 25-30%, 3.7-4.5 min: 30%, 4.5-6 min: 30-40%, 6-7 min: 40-90%, 7-8 min: 95%. The flow rate was 0.5 mL/min and the injection volume was 1 μL.

Mass spectrum AB Sciex 6500 plus QTRAP (ESI-MS/MS) employed a positive ion mode with ion source (chamber) conditions as follows: air curtain gas pressure 35 psi, collision cell gas flow selection medium, ion spray voltage 4500 V, spray gas pressure 55 psi, spray gas temperature 400° C., and auxiliary heater gas pressure 50 psi. The scanning mode was scheduled multiple reaction monitoring (sMRM) mode. The common daughter ion of light-labeled derivatization product was m/z 142. 2, and that of heavy-labeled derivatization product is m/z 144. 2. The collision energy (CE) of each derivatization product was respectively optimized upon derivatization under each derivatization standard.

### Test Results 1 Linear Range, Correlation Coefficient, and Limit of Quantification of 65 Nucleoside Metabolites

DELANS-Cl was reacted with the working solutions of various concentration gradients to obtain the linear ranges, linear correlation coefficients, and minimum quantitation limits of 65 nucleoside metabolites.

TABLE 3
Linear range, linear correlation coefficient, and limit of quantification of 65 nucleoside metabolites

| | | | Limit of | Linear range (nM) | | Linear |
|---|---|---|---|---|---|---|
| | | | quantitation | Lower | Upper | correlation |
| No. | Metabolite | Linear equation | LOQ (fmol) | limit | limit | coefficient (R² [symbol] |
| 1 | Adenine | y = 0.0091x + 0.0116 | 0.198 | 0.182 | 729.345 | 0.998 |
| 2 | Guanine | y = 0.0118x + 0.0129 | 0.203 | 0.182 | 729.345 | 0.997 |
| 3 | Hypoxanthine | y = 0.0122x + 0.0049 | 0.246 | 0.182 | 729.345 | 0.996 |
| 4 | Xanthine | y = 0.0078x + 0.0131 | 0.045 | 0.182 | 729.345 | 0.998 |
| 5 | Uracil | y = 0.0078x + 0.0138 | 0.032 | 0.274 | 1094.017 | 0.998 |
| 6 | Cytosine | y = 0.0226x + 0.5196 | 0.517 | 1.823 | 729.345 | 0.997 |
| 7 | Thymine | y = 0.0185x + 0.0060 | 0.079 | 0.182 | 729.345 | 0.997 |
| 8 | Adenosine | y = 0.0202x + 0.0164 | 0.521 | 0.182 | 729.345 | 0.997 |
| 9 | Guanosine | y = 0.0174x + 0.0247 | 0.286 | 0.252 | 504.123 | 0.998 |
| 10 | Uridine | y = 0.0033x + 0.0084 | 1.216 | 0.912 | 3646.724 | 0.997 |
| 11 | Cytidine | y = 0.0175x + 0.2168 | 0.368 | 0.365 | 729.345 | 0.997 |
| 12 | Thymidine | y = 0.0228x + 0.1164 | 1.351 | 0.729 | 729.345 | 0.995 |
| 13 | Inosine | y = 0.0151x + 0.0418 | 0.351 | 0.182 | 729.345 | 0.996 |
| 14 | Xanthosine | y = 0.0165x + 0.0743 | 0.445 | 0.365 | 729.345 | 0.998 |
| 15 | Deoxyadenosine | y = 0.0260x + 0.7010 | 1.057 | 0.729 | 729.345 | 0.997 |
| 16 | Deoxyguanosine | y = 0.0290x + 0.0036 | 0.217 | 0.182 | 729.345 | 0.995 |
| 17 | Deoxyuridine | y = 0.0278x + 0.0619 | 2.224 | 1.823 | 729.345 | 0.997 |
| 18 | Deoxycytidine | y = 0.0711x + 0.1102 | 3.039 | 1.823 | 729.345 | 0.997 |
| 19 | Deoxyinosine | y = 0.0130x + 0.0007 | 0.090 | 0.182 | 729.345 | 0.997 |
| 20 | 1-methyladenosine | y = 0.0154x + 0.0056 | 0.109 | 0.182 | 729.345 | 0.999 |
| 21 | 2-methyladenosine | y = 0.0220x + 0.1307 | 0.038 | 0.182 | 729.345 | 0.997 |
| 22 | N⁶-methyladenosine | y = 0.0206x + 0.0041 | 0.200 | 0.182 | 729.345 | 0.997 |
| 23 | 2′-O-methyladenosine | y = 0.0248x + 0.3439 | 7.597 | 2.735 | 729.345 | 0.995 |
| 24 | N⁶,2′-O-dimethyladenosine | y = 0.0075x + 0.0117 | 1.552 | 0.729 | 729.345 | 0.998 |
| 25 | N¹,2′-O-dimethyladenosine | y = 0.0267x + 2.6341 | 1.614 | 1.823 | 729.345 | 0.997 |
| 26 | 3′-O-methyladenosine | y = 0.0431x + 0.0202 | 6.078 | 1.823 | 729.345 | 0.997 |
| 27 | 8-methyladenosine | y = 0.0222x + 0.0046 | 0.450 | 0.365 | 729.345 | 0.998 |
| 28 | 3-methylcytidine | y = 0.0133x + 0.0019 | 0.314 | 0.182 | 729.345 | 0.995 |
| 29 | N⁴-methylcytidine | y = 0.0220x + 0.0898 | 3.090 | 1.823 | 729.345 | 0.999 |
| 30 | 5-methylcytidine | y = 0.0246x + 0.0033 | 3.171 | 0.729 | 729.345 | 0.997 |
| 31 | 5-hydroxymethylcytidine | y = 0.0217x + 0.3079 | 2.279 | 1.823 | 729.345 | 0.997 |
| 32 | 2′-O-methylcytidine | y = 0.0013x + 0.0091 | 4.144 | 1.823 | 729.345 | 0.998 |
| 33 | N⁴,2′-O-dimethylcytidine | y = 0.0460x + 0.0256 | 7.597 | 1.823 | 729.345 | 0.999 |
| 34 | 2′-C-methylcytidine | y = 0.0219x + 0.0017 | 8.481 | 3.647 | 729.345 | 0.998 |
| 35 | 5-methyl-2′-O-methylcytidine | y = 0.0076x + 0.2137 | 9.910 | 9.117 | 729.345 | 0.996 |
| 36 | 1-methylguanosine | y = 0.0215x + 0.0022 | 0.145 | 0.182 | 729.345 | 0.997 |
| 37 | 2-methylguanosine | y = 0.0249x + 0.0083 | 0.374 | 0.292 | 729.345 | 0.995 |
| 38 | N²,N²-dimethylguanosine | y = 0.0133x + 0.0208 | 7.013 | 1.823 | 729.345 | 0.996 |
| 39 | 7-methylguanosine | y = 0.0107x + 0.0044 | 0.414 | 0.182 | 729.345 | 0.998 |
| 40 | 2′-O-methylguanosine | y = 0.0310x + 0.0078 | 0.424 | 0.182 | 729.345 | 0.996 |
| 41 | 5-methyluridine | y = 0.0184x + 0.0042 | 0.264 | 0.182 | 729.345 | 0.997 |
| 42 | 2′-O-methyluridine | y = 0.0323x + 0.0051 | 1.140 | 0.365 | 729.345 | 0.998 |
| 43 | 5-methyl-2′-O-methyluridine | y = 0.0358x + 0.0886 | 0.986 | 0.729 | 729.345 | 0.996 |
| 44 | 2′-O-methoxyinosine | y = 0.0136x + 0.0115 | 0.038 | 0.182 | 729.345 | 0.997 |
| 45 | 5-methyldeoxycytidine | y = 0.0077x + 0.1500 | 0.506 | 0.365 | 729.345 | 0.997 |
| 46 | 5-hydroxymethyldeoxycytidine | y = 0.0039x + 0.0874 | 17.532 | 9.117 | 729.345 | 0.998 |
| 47 | N⁶-methyldeoxyadenosine | y = 0.0012x + 0.0048 | 40.519 | 18.234 | 729.345 | 0.997 |
| 48 | 3-methyladenine | y = 0.0306x + 0.0612 | 0.611 | 0.201 | 729.345 | 0.999 |
| 49 | 5′-methylcytosine | y = 0.0207x + 0.1625 | 0.157 | 0.365 | 729.345 | 0.996 |
| 50 | 5-hydroxymethylcytosine | y = 0.0164x + 0.0390 | 1.403 | 0.547 | 729.345 | 0.998 |
| 51 | 5-formylcytosine | y = 0.0089x + 0.0314 | 0.744 | 0.365 | 729.345 | 0.999 |
| 52 | 5-carboxylcytosine | y = 0.0135x + 0.0632 | 0.257 | 1.094 | 729.345 | 0.996 |
| 53 | 7-methylguanine | y = 0.0042x + 0.0397 | 3.721 | 1.823 | 729.345 | 0.997 |
| 54 | 5,6-dihydrouracil | y = 0.0130x + 0.0192 | 0.276 | 0.182 | 729.345 | 0.998 |
| 55 | 2-aminoadenosine | y = 0.0229x + 0.0007 | 0.829 | 0.365 | 729.345 | 0.998 |
| 56 | 8-aminoadenosine | y = 0.0140x + 0.0005 | 0.445 | 0.182 | 729.345 | 0.999 |
| 57 | 8-oxoadenosine | y = 0.0126x + 0.0105 | 0.536 | 0.182 | 729.345 | 0.999 |
| 58 | Dihydrouridine | y = 0.0237x + 0.0040 | 0.368 | 0.365 | 729.345 | 0.998 |
| 59 | Pseudouridine | y = 0.0105x + 0.0009 | 0.068 | 0.182 | 729.345 | 0.997 |
| 60 | Orotidine | y = 0.0023x + 0.0055 | 6.181 | 3.647 | 729.345 | 0.996 |
| 61 | 5-methoxycarbonylmethyluridine | y = 0.0214x + 0.0003 | 0.376 | 0.365 | 729.345 | 0.998 |
| 62 | 5-aminomethylcarbamoylmethyluridine | y = 0.0359x + 0.0285 | 1.488 | 0.729 | 729.345 | 0.998 |
| 63 | 5-methoxycarbonylmethyl-2-thiouridine | y = 0.0026x + 0.0303 | 52.096 | 18.234 | 729.345 | 0.995 |
| 64 | 5-methoxycarbonylmethyl-2′-O- | y = 0.0260x + 0.0376 | 0.285 | 0.182 | 729.345 | 0.997 |
| | methyluridine | | | | | |
| 65 | N⁴-acetylcytidine | y = 0.0295x + 0.0021 | 0.557 | 0.306 | 729.345 | 0.997 |

[symbol]

indicates data missing or illegible when filed

### Test Results 2

The results of sensitivity comparison of derivatization reaction of DELANS-Cl with DNS-Cl and DENS-Cl are listed in Table 4.

TABLE 4
DELANS-Cl vs DNS-Cl and DENS-Cl derivatization sensitivity comparison

| | | | | | DELANS-Cl | DELANS-Cl |
|---|---|---|---|---|---|---|
| | | LOQ for | LOQ for | LOQ for | vs DNS-Cl | vs DENS-Cl |
| | | DELANS-Cl | DNS-Cl | DENS-Cl | sensitivity | sensitivity |
| | | method | method | method | improvement | enhancement |
| No. | Metabolite | (fmol) | (fmol) | (fmol) | factor | factor |
| 1 | Adenine | 0.198 | 0.148 | 0.289 | 0.75 | 1.46 |
| 2 | Guanine | 0.203 | 12.662 | 6.119 | 62.50 | 30.20 |
| 3 | Hypoxanthine | 0.246 | 6.907 | 3.453 | 28.03 | 14.02 |
| 4 | Xanthine | 0.045 | 9.117 | 5.180 | 203.00 | 115.34 |
| 5 | Uracil | 0.032 | 0.810 | 0.595 | 25.03 | 18.37 |
| 6 | Cytosine | 0.517 | 2.650 | 13.898 | 5.13 | 26.91 |
| 7 | Thymine | 0.079 | 0.556 | 0.289 | 7.01 | 3.64 |
| 8 | Adenosine | 0.521 | 15.195 | 2.849 | 29.17 | 5.47 |
| 9 | Guanosine | 0.286 | 1.575 | 2.864 | 5.50 | 10.00 |
| 10 | Uridine | 1.216 | 11.748 | 1.521 | 9.66 | 1.25 |
| 11 | Cytidine | 0.368 | 3.506 | 5.698 | 9.52 | 15.47 |
| 12 | Thymidine | 1.351 | 42.207 | 4.604 | 31.25 | 3.41 |
| 13 | Inosine | 0.351 | 189.938 | 22.237 | 541.68 | 63.42 |
| 14 | Xanthosine | 0.445 | 3.506 | 5.698 | 7.88 | 12.81 |
| 15 | Deoxyadenosine | 1.057 | 10.130 | 25.682 | 9.58 | 24.30 |
| 16 | Deoxyguanosine | 0.217 | 1.302 | 2.192 | 6.00 | 10.10 |
| 17 | Deoxyuridine | 2.224 | 15.452 | 7.473 | 6.95 | 3.36 |
| 18 | Deoxycytidine | 3.039 | 25.324 | 112.000 | 8.33 | 36.86 |
| 19 | Deoxyinosine | 0.090 | 2.374 | 2.590 | 26.43 | 28.84 |
| 20 | 1-methyladenosine | 0.109 | 0.127 | 0.375 | 1.17 | 3.46 |
| 21 | 2-methyladenosine | 0.038 | 2.730 | 8.082 | 72.75 | 215.43 |
| 22 | N⁶-methyladenosine | 0.200 | 1.266 | 1.190 | 6.32 | 5.94 |
| 23 | 2′-O-methyladenosine | 7.597 | 14.245 | 43.831 | 1.88 | 5.77 |
| 24 | N⁶,2′-O-dimethyladenosine | 1.552 | 6.512 | 21.104 | 4.20 | 13.60 |
| 25 | N¹,2′-O-dimethyladenosine | 1.614 | 7.597 | 22.792 | 4.71 | 14.13 |
| 26 | 3′-O-methyladenosine | 6.078 | 15.195 | 7.597 | 2.50 | 1.25 |
| 27 | 8-methyladenosine | 0.450 | 0.684 | 0.829 | 1.52 | 1.84 |
| 28 | 3-methylcytidine | 0.314 | 0.712 | 1.952 | 2.27 | 6.21 |
| 29 | N⁴-methylcytidine | 3.090 | 3.618 | 7.236 | 1.17 | 2.34 |
| 30 | 5-methylcytidine | 3.171 | 3.506 | 35.753 | 1.11 | 11.27 |
| 31 | 5-hydroxymethylcytidine | 2.279 | 4.341 | 10.130 | 1.90 | 4.44 |
| 32 | 2′-O-methylcytidine | 4.144 | 18.993 | 33.518 | 4.58 | 8.09 |
| 33 | N⁴,2′-O-dimethylcytidine | 7.597 | 1.302 | 172.340 | 0.17 | 22.68 |
| 34 | 2′-C-methylcytidine | 8.481 | 2.279 | 10.244 | 0.27 | 1.21 |
| 35 | 5-methyl-2′-O-methylcytidine | 9.910 | 45.584 | 223.451 | 4.60 | 22.55 |
| 36 | 1-methylguanosine | 0.145 | 0.190 | 0.570 | 1.31 | 3.94 |
| 37 | 2-methylguanosine | 0.374 | 18.990 | 10.483 | 50.77 | 28.03 |
| 38 | N²,N²-dimethylguanosine | 7.013 | 4.798 | 20.720 | 0.68 | 2.95 |
| 39 | 7-methylguanosine | 0.414 | 0.190 | 1.140 | 0.46 | 2.75 |
| 40 | 2′-O-methylguanosine | 0.424 | 1.302 | 0.912 | 3.07 | 2.15 |
| 41 | 5-methyluridine | 0.264 | 4.144 | 1.216 | 15.68 | 4.60 |
| 42 | 2′-O-methyluridine | 1.140 | 14.245 | 13.024 | 12.50 | 11.43 |
| 43 | 5-methyl-2′-O-methyluridine | 0.986 | 8.140 | 1.349 | 8.26 | 1.37 |
| 44 | 2′-O-methoxyinosine | 0.038 | 2.171 | 1.212 | 57.62 | 32.18 |
| 45 | 5-methyldeoxycytidine | 0.506 | 45.584 | 113.963 | 90.00 | 225.00 |
| 46 | 5-hydroxymethyldeoxycytidine | 17.532 | 63.311 | 284.900 | 3.61 | 16.25 |
| 47 | N⁶-methyldeoxyadenosine | 40.519 | 5.534 | 11.395 | 0.14 | 0.28 |
| 48 | 3-methyladenine | 0.611 | 4.131 | 97.620 | 6.77 | 159.89 |
| 49 | 5′-methylcytosine | 0.157 | 1.727 | 9.027 | 10.98 | 57.43 |
| 50 | 5-hydroxymethylcytosine | 1.403 | 5.698 | 8.339 | 4.06 | 5.95 |
| 51 | 5-formylcytosine | 0.744 | 12.662 | 7.597 | 17.01 | 10.21 |
| 52 | 5-carboxylcytosine | 0.257 | 19.877 | 37.500 | 77.40 | 146.02 |
| 53 | 7-methylguanine | 3.721 | 21.104 | 2.952 | 5.67 | 0.79 |
| 54 | 5,6-dihydrouracil | 0.276 | 21.707 | 19.648 | 78.57 | 71.12 |
| 55 | 2-aminoadenosine | 0.829 | 1.530 | 4.240 | 1.85 | 5.12 |
| 56 | 8-aminoadenosine | 0.445 | 1.838 | 2.903 | 4.13 | 6.53 |
| 57 | 8-oxoadenosine | 0.536 | 18.381 | 27.460 | 34.27 | 51.20 |
| 58 | Dihydrouridine | 0.368 | 2.399 | 11.396 | 6.51 | 30.94 |
| 59 | Pseudouridine | 0.068 | 0.414 | 1.249 | 6.09 | 18.36 |
| 60 | Orotidine | 6.181 | 1.599 | 9.117 | 0.26 | 1.48 |
| 61 | 5-methoxycarbonylmethyluridine | 0.376 | 5.698 | 0.166 | 15.16 | 0.44 |
| 62 | 5-aminomethylcarbamoylmethyluridine | 1.488 | 9.402 | 2.600 | 6.32 | 1.75 |
| 63 | 5-methoxycarbonylmethyl-2-thiouridine | 52.096 | 2170.667 | 75.973 | 41.67 | 1.46 |
| 64 | 5-methoxycarbonylmethyl-2′-O-methyluridine | 0.285 | 24.508 | 7.859 | 86.02 | 27.59 |
| 65 | N⁴-acetylcytidine | 0.557 | 9.190 | 5.698 | 16.50 | 10.23 |

By comparison between the improved sensitivities of the derivatization reagent DELANS-Cl of the present disclosure and the commercial derivatization reagent DNS-Cl, it can be seen that the sensitivities of more than 58 metabolites in the nucleoside metabolite library (65) are improved. Compared with DNS-Cl, the derivatization reagent DELANS-Cl of the present disclosure improves the sensitivity up to 541 times. Compared with DENS-Cl, the derivatization reagent DELANS-Cl of the present disclosure increases the sensitivity up to 225 times.

### Test Results 3

The derivatization reagent DELANS-Cl can be used to derivatize amino- and hydroxyl-containing metabolites (i.e. nucleoside metabolites) in urine, serum, tissue and cell samples. Upon derivatization, the derivatized samples can be quantitatively detected by ultra performance liquid chromatography-mass spectrometry (UHPLC-MS/MS) to detect 58, 55, 59 and 60 nucleoside metabolites, respectively. The analysis results are listed in Table 5.

TABLE 5
Detection results of nucleoside metabolites in urine, serum, tissue, and cell samples

| | | Urine | Serum | Tissue | Cell |
|---|---|---|---|---|---|
| No. | Metabolite | (10⁻¹ μM) | (10⁻¹ μM) | (10⁻² nmol/mg) | (10⁻² nmol/mg) |
| 1 | Adenine | 122.0 ± 5.7 | 104.4 ± 4.1 | 52.8 ± 5.8 | 147.4 ± 8.3 |
| 2 | Guanine | 89.4 ± 7.9 | ND | 197 ± 10.2 | 133.7 ± 8.8 |
| 3 | Hypoxanthine | 207.5 ± 17.8 | 13.9 ± 1.4 | 875.8 ± 80.3 | 91.9 ± 10.6 |
| 4 | Xanthine | 414.2 ± 16.8 | 8.4 ± 0.9 | 1774.2 ± 87.4 | 145.1 ± 9.4 |
| 5 | Uracil | 386.8 ± 15.4 | 0.8 ± 0.1 | 324.1 ± 10.6 | 149.0 ± 5.9 |
| 6 | Cytosine | 0.1 ± 0.0 | 0.1 ± 0.0 | 237.9 ± 25.4 | 11.0 ± 1.0 |
| 7 | Thymine | 10.8 ± 0.5 | 0.7 ± 0.2 | 3.2 ± 0.5 | 2.4 ± 0.3 |
| 8 | Adenosine | 23.1 ± 3.2 | 1.4 ± 0.4 | 2269.4 ± 175.1 | 2231.5 ± 187.0 |
| 9 | Guanosine | 3.1 ± 1.1 | 0.1 ± 0.0 | 3261.8 ± 294.3 | 2121 ± 216.6 |
| 10 | Uridine | 11.6 ± 2.0 | 16.6 ± 1.6 | 2551.2 ± 103.7 | 1995.9 ± 100.0 |
| 11 | Cytidine | 15.1 ± 2.6 | ND | 2498 ± 302.3 | 2017.9 ± 201.6 |
| 12 | Thymidine | 15.6 ± 4.4 | 63.1 ± 12.4 | 18979.1 ± 1308.5 | 13179.1 ± 1847.5 |
| 13 | Inosine | 19.4 ± 4.6 | 2.8 ± 1.4 | 2801.6 ± 241.1 | 1248.2 ± 117.6 |
| 14 | Xanthosine | 34.6 ± 7.7 | 5.0 ± 1.7 | 40.6 ± 9.5 | ND |
| 15 | Deoxyadenosine | ND | 1292.8 ± 247.1 | 8450.8 ± 580.0 | 4144.2 ± 329.0 |
| 16 | Deoxyguanosine | 121.4 ± 26.2 | 14.1 ± 2.3 | 844.1 ± 91.5 | 1069.5 ± 104.1 |
| 17 | Deoxyuridine | 14.4 ± 5.3 | 11.0 ± 3.4 | 687.7 ± 92.0 | 99.7 ± 10.5 |
| 18 | Deoxycytidine | ND | 27.6 ± 8.4 | 571.5 ± 114.5 | 363.7 ± 47.3 |
| 19 | Deoxyinosine | 0.3 ± 0.1 | 0.1 ± 0.1 | 168.8 ± 18.4 | 3.4 ± 0.4 |
| 20 | 1-methyladenosine | 110.4 ± 7.7 | 0.2 ± 0.1 | 24.4 ± 2.2 | 15.2 ± 0.9 |
| 21 | 2-methyladenosine | 4.9 ± 3.7 | 2.5 ± 1.4 | 11.4 ± 1.7 | 1.1 ± 0.1 |
| 22 | N⁶-methyladenosine | 1.0 ± 0.2 | 0.4 ± 0.2 | 5.8 ± 1.5 | 5.9 ± 1 |
| 23 | 2′-O-methyladenosine | 113.7 ± 17.7 | 8.1 ± 3.6 | 6650.6 ± 926.3 | 1953.2 ± 177.9 |
| 24 | N⁶,2′-O-dimethyladenosine | 25.5 ± 10.6 | 7.3 ± 1.7 | 616.5 ± 51.2 | 118.9 ± 8.1 |
| 25 | N¹,2′-O-dimethyladenosine | 129.6 ± 47.8 | 1.4 ± 0.0 | 1638.1 ± 185.9 | 496.4 ± 65.1 |
| 26 | 3′-O-methyladenosine | 4.7 ± 0.9 | 3.1 ± 2.1 | 176.5 ± 18.3 | 31.6 ± 5.5 |
| 27 | 8-methyladenosine | 12.7 ± 7.2 | 4.2 ± 2.2 | 42.1 ± 6.0 | 27.9 ± 4.7 |
| 28 | 3-methylcytidine | 34.8 ± 4.0 | 0.4 ± 0.2 | 36.4 ± 6.1 | 5.4 ± 1.0 |
| 29 | N⁴-methylcytidine | ND | ND | ND | 16.1 ± 8.1 |
| 30 | 5-methylcytidine | 9.3 ± 3.7 | 3.2 ± 1.4 | 21.3 ± 4.1 | 13.6 ± 3.1 |
| 31 | 5-hydroxymethylcytidine | 14.1 ± 4.7 | 26.916.9 | 1154 ± 108.8 | 404.4 ± 39.0 |
| 32 | 2′-O-methylcytidine | 632.7 ± 73.9 | 9.4 ± 5.7 | 20587.7 ± 2113.8 | 7091.8 ± 685.8 |
| 33 | N⁴,2′-O-dimethylcytidine | 18.6 ± 11.3 | 7.8 ± 2.0 | ND | ND |
| 34 | 2′-C-methylcytidine | 13.7 ± 2.2 | 4.4 ± 1.7 | ND | 12.4 ± 2.7 |
| 35 | 5-methyl-2′-O-methylcytidine | ND | ND | 311.5 ± 92.7 | 54.2 ± 17.4 |
| 36 | 1-methylguanosine | 33.7 ± 4.5 | 0.3 ± 0.1 | 14.3 ± 2.9 | 7.5 ± 1.1 |
| 37 | 2-methylguanosine | 8.2 ± 1.0 | ND | 1.8 ± 1.4 | 0.8 ± 0.2 |
| 38 | N²,N²-dimethylguanosine | 217.5 ± 28.5 | 2.2 ± 0.6 | 26.4 ± 5.3 | 10.6 ± 1.9 |
| 39 | 7-methylguanosine | 0.9 ± 0.3 | 0.2 ± 0.1 | 22.6 ± 2.1 | 12.4 ± 1.2 |
| 40 | 2′-O-methylguanosine | 5.8 ± 1.0 | 0.9 ± 0.2 | 38.4 ± 12.3 | 25.3 ± 5.1 |
| 41 | 5-methyluridine | 1.1 ± 0.8 | 1.9 ± 1.1 | 73.4 ± 6.1 | 35.1 ± 5.3 |
| 42 | 2′-O-methyluridine | 6 ± 2.8 | 1.8 ± 0.5 | 110.3 ± 19.9 | 76.6 ± 16 |
| 43 | 5-methyl-2′-O-methyluridine | 12.1 ± 2.5 | 5.4 ± 1.4 | 357.6 ± 101.3 | 32.1 ± 8.2 |
| 44 | 2′-O-methoxyinosine | 4.5 ± 0.5 | 0.7 ± 0.2 | 11.2 ± 1.8 | 3.4 ± 0.6 |
| 45 | 5-methyldeoxycytidine | 13.3 ± 4 | 1.2 ± 0.0 | 63.3 ± 9.3 | 10.4 ± 1.5 |
| 46 | 5-hydroxymethyldeoxycytidine | 11.8 ± 0.0 | ND | ND | ND |
| 47 | N⁶-methyldeoxyadenosine | ND | ND | 5938.6 ± 866.8 | 2490.8 ± 520.0 |
| 48 | 3-methyladenine | 30.6 ± 13.0 | 8.6 ± 1.0 | 330.0 ± 35.0 | 85.6 ± 9.8 |
| 49 | 5′-methylcytosine | ND | ND | 454.5 ± 43.3 | 346.8 ± 33.1 |
| 50 | 5-hydroxymethylcytosine | ND | 6.6 ± 3.5 | 328.0 ± 43.6 | 355.4 ± 51.5 |
| 51 | 5-formylcytosine | 323.3 ± 25.6 | 27.7 ± 3.7 | 192.8 ± 23.8 | ND |
| 52 | 5-carboxylcytosine | 284.2 ± 53.6 | 82.8 ± 15.8 | 1522.8 ± 299.6 | 293.3 ± 55.5 |
| 53 | 7-methylguanine | 5895.5 ± 737.6 | 20.9 ± 4.8 | 497.9 ± 109.9 | 276.3 ± 91.0 |
| 54 | 5,6-dihydrouracil | 254.7 ± 9.0 | 0.1 ± 0.1 | 243.3 ± 37.1 | 107.7 ± 7.9 |
| 55 | 2-aminoadenosine | 8.3 ± 2.9 | 1.9 ± 1.3 | ND | 2.3 ± 0.5 |
| 56 | 8-aminoadenosine | 2.3 ± 0.5 | 0.5 ± 0.3 | 9.5 ± 1.9 | 5.8 ± 1.4 |
| 57 | 8-oxoadenosine | 7.2 ± 2.1 | 1.8 ± 0.6 | 4867.6 ± 491.2 | 3239.8 ± 331.7 |
| 58 | Dihydrouridine | 284.6 ± 30.7 | 5.1 ± 1.7 | 171.6 ± 28.1 | 46.3 ± 7.3 |
| 59 | Pseudouridine | 1369.9 ± 59.8 | 15 ± 1.7 | 260.3 ± 15.5 | 154.4 ± 9.3 |
| 60 | Orotidine | 30 ± 12.5 | ND | ND | ND |
| 61 | 5-methoxycarbonylmethyluridine | 3.2 ± 1.3 | 0.4 ± 0.2 | 34.7 ± 9.0 | 23.2 ± 5.9 |
| 62 | 5-aminomethylcarbamoylmethyluridine | 21.1 ± 3.6 | 2.2 ± 0.8 | 339.0 ± 55.1 | 181.1 ± 38.8 |
| 63 | 5-methoxycarbonylmethyl-2-thiouridine | 51.8 ± 11.0 | ND | 63.7 ± 50.0 | 41.2 ± 6.1 |
| 64 | 5-methoxycarbonylmethyl-2′-O- | 10.4 ± 1.3 | 1.5 ± 0.8 | 999.2 ± 193.5 | 121.3 ± 25.8 |
| | methyluridine | | | | |
| 65 | N⁴-acetylcytidine | 24.2 ± 6.2 | 2.5 ± 1.0 | 146.0 ± 24.2 | 97.1 ± 17.9 |

Comments: “nd” means “not detected” or “below the limit of quantitation”.

### Test Result 4

TABLE 6
Retention time of working solution S₂ and metabolites in
working solution S₂ upon derivatization with DELANS-Cl

| | Retention time without | Retention time upon |
|---|---|---|
| | derivatization (min) | derivatization (min) |
| Adenine | 1.021 | 4.402 |
| Guanine | 1.052 | 3.298 |
| Hypoxanthine | 1.023 | 4.149 |
| Xanthine | 1.040 | 2.610 |
| Uracil | 1.047 | 4.631 |
| Cytosine | 1.047 | 3.084 |
| Thymine | 0.544 | 5.646 |
| Adenosine | 0.761 | 1.970 |
| Guanosine | 2.099 | 1.921 |
| Uridine | 2.495 | 3.004 |
| Cytidine | 1.080 | 1.835 |
| Thymidine | 1.083 | 4.979 |
| Inosine | 2.250 | 1.981 |
| Xanthosine | 2.001 | 2.053 |
| Deoxyadenosine | 2.866 | 4.065 |
| Deoxyguanosine | 2.474 | 3.835 |
| Deoxyuridine | 2.623 | 4.534 |
| Deoxycytidine | 2.388 | 3.791 |
| Deoxyinosine | 1.408 | 4.179 |
| 1-methyladenosine | 1.493 | 1.491 |
| 2-methyladenosine | 2.014 | 2.001 |
| N⁶-methyladenosine | 1.991 | 2.441 |
| 2′-O-methyladenosine | 3.206 | 4.790 |
| N⁶,2′-O-dimethyladenosine | 2.961 | 5.615 |
| N¹,2′-O-dimethyladenosine | 2.961 | 2.484 |
| 3′-O-methyladenosine | 2.432 | 4.407 |
| 8-methyladenosine | 2.011 | 2.275 |
| 3-methylcytidine | 1.621 | 1.574 |
| N⁴-methylcytidine | 1.418 | 1.982 |
| 5-methylcytidine | 1.440 | 1.881 |
| 5-hydroxymethylcytidine | 1.437 | 1.840 |
| 2′-O-methylcytidine | 2.663 | 2.681 |
| N⁴,2′-O-dimethylcytidine | 2.323 | 3.036 |
| 2′-C-methylcytidine | 2.477 | 1.911 |
| 5-methyl-2′-O-methylcytidine | 1.173 | 2.861 |
| 1-methylguanosine | 2.039 | 2.041 |
| 2-methylguanosine | 2.061 | 2.752 |
| N²,N²-dimethylguanosine | 2.201 | 2.371 |
| 7-methylguanosine | 2.173 | 1.441 |
| 2′-O-methylguanosine | 3.117 | 4.511 |
| 5-methyluridine | 3.197 | 3.480 |
| 2′-O-methyluridine | 2.718 | 4.831 |
| 5-methyl-2′-O-methyluridine | 2.949 | 5.270 |
| 2′-O-methoxyinosine | 3.820 | 4.851 |
| 5-methyldeoxycytidine | 1.081 | 2.441 |
| 5-hydroxymethyldeoxycytidine | 1.086 | 1.795 |
| N⁶-methyldeoxyadenosine | 1.170 | 3.181 |
| 3-methyladenine | 3.303 | 5.558 |
| 5′-methylcytosine | 0.756 | 5.274 |
| 5-hydroxymethylcytosine | 0.526 | 2.596 |
| 5-formylcytosine | 0.408 | 5.533 |
| 5-carboxylcytosine | 1.442 | 4.632 |
| 7-methylguanine | 1.727 | 4.841 |
| 5,6-dihydrouracil | 1.075 | 4.690 |
| 2-aminoadenosine | 1.318 | 1.636 |
| 8-aminoadenosine | 1.318 | 1.551 |
| 8-oxoadenosine | 1.457 | 1.844 |
| Dihydrouridine | 1.089 | 2.631 |
| Pseudouridine | 1.111 | 2.132 |
| Orotidine | 1.894 | 2.515 |
| 5-methoxycarbonylmethyluridine | 3.346 | 3.852 |
| 5-aminomethylcarbamoylmethyluridine | 1.925 | 2.556 |
| 5-methoxycarbonylmethyl-2- | 4.499 | 5.248 |
| thiouridine | | |
| 5-methoxycarbonylmethyl-2′-O- | 4.433 | 5.318 |
| methyluridine | | |
| N⁴-acetylcytidine | 3.375 | 3.533 |

By comparison, partially underivatized metabolites have poor retention on the chromatographic column (retention time close to the dead volume of the column, about 0.5 min), and metabolites with poor retention on the chromatographic column were improved after derivatization, with retention time within 3-6 min.

What is claimed is:

1. A compound of formula (I) or a salt thereof: 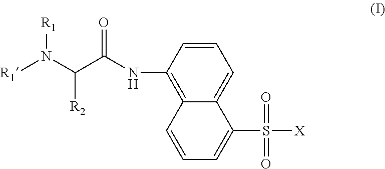
wherein R₁ and R_{1′} are independently selected from C₁₋₇ alkyl;
R₂ is selected from H, C₁₋₇ alkyl, or benzyl; and
X is selected from OH or halogen.

2. The compound of formula (I) or the salt thereof according to claim 1, wherein R₁ and R_{1′} are the same.

3. The compound of formula (I) or the salt thereof according to claim 1, wherein,
in R₁ or R_{1′}, the C₁₋₇ alkyl is C₁₋₄ alkyl; and/or
in R₂, the C₁₋₇ alkyl is C₁₋₄ alkyl; and/or
in X, the halogen is Cl.

4. The compound of formula (I) or the salt thereof according to claim 3, wherein the C₁₋₇ alkyl in R₁ or R_{1′} is C₂₋₄ alkyl or ethyl.

5. The compound of formula (I) or the salt thereof according to claim 3, wherein the C₁₋₇ alkyl in R₂ is isobutyl.

6. The compound of formula (I) or the salt thereof according to claim 1, wherein the compound of the formula (I) is

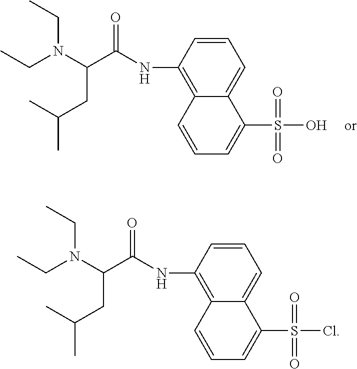 [SMILES: CCN(CC)C(CC(C)C)C(=O)Nc1cccc2c(S(=O)(=O)O)cccc12 ; CCN(CC)C(CC(C)C)C(=O)Nc1cccc2c(S(=O)(=O)Cl)cccc12]

7. The compound of formula (I) or the salt thereof according to claim 1, wherein the salt of the compound of formula (I) is a salt obtained from the compound of formula (I) and an acid, and the acid is an inorganic acid or an organic acid.

8. A preparation method for a compound of formula (I), comprising:
subjecting a compound of formula (III) to a condensation reaction with a compound of formula (IV) in the presence of an activating agent and a base in a solvent to obtain the compound of formula (I): 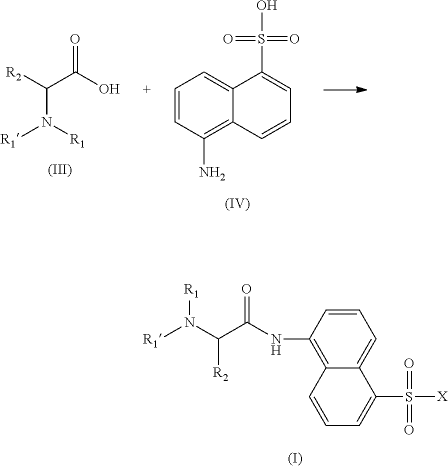
wherein, X is OH, R₁ and R_{1′} are independently selected from C₁₋₇ alkyl, and R₂ is selected from H, C₁₋₇ alkyl, or benzyl.

9. The preparation method for the compound of formula (I) according to claim 8, wherein,
in the condensation reaction, the solvent is N,N-dimethylformamide (DMF); and/or
in the condensation reaction, the activating agent comprises one or more of 4-(4,6-dimethoxytriazin-2-yl)-4-methylmorpholine hydrochloride, 1-(3-dimethylaminopropyl)-3-ethylcarbodiimide hydrochloride and 1-hydroxybenzotriazole; and/or
in the condensation reaction, the base is an organic base.

10. The preparation method for the compound of formula (I) according to claim 9, wherein the organic base comprises N-methylmorpholine and/or pyridine.

11. The preparation method for the compound of formula (I) according to claim 8, further comprising a process (1-1) or a process (1-2); wherein
the process (1-1) comprises: subjecting a compound of formula (VI) to a reductive amination reaction with a compound of formula (A-1) and a compound of formula (A-2) in the presence of a reducing agent in a solvent to obtain the compound of formula (III): 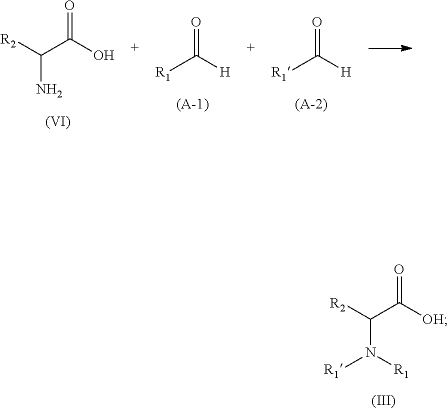
the process (1-2) comprises: subjecting a compound of formula (VI) to an alkylation reaction with a compound of formula (B-1) and a compound of formula (B-2) in the presence of a base in a solvent to obtain the compound of formula (III): 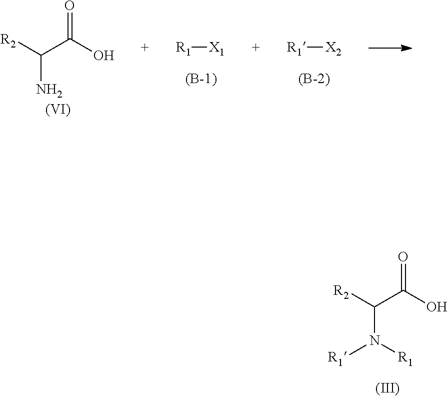
wherein X₁ and X₂ are independently halogen.

12. The preparation method for the compound of formula (I) according to claim 11, wherein, in the process (1-2), the halogen is I.

13. The preparation method for the compound of formula (I) according to claim 11, wherein,
in the reductive amination reaction, the solvent is methanol, acetonitrile, or a buffer of sodium acetate or phosphate with a pH of 2-12; and/or
in the reductive amination reaction, the reducing agent is sodium cyanoborohydride and/or 2-methylpyridine borane; and/or
in the alkylation reaction, the solvent is acetonitrile; and/or
in the alkylation reaction, the base is carbonate or bicarbonate.

14. The preparation method for the compound of formula (I) according to claim 13, wherein the carbonate is a potassium carbonate.

15. A preparation method for a compound of formula (I), comprising:
subjecting a compound of formula (III) to a condensation reaction with a compound of formula (IV) in the presence of an activating agent and a base in a solvent to obtain a compound of formula (V): 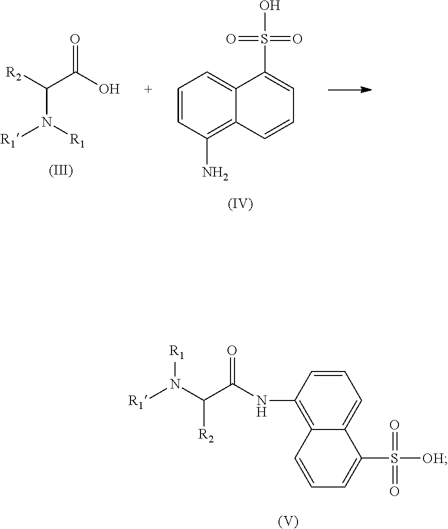
and
subjecting the compound of formula (V) to an acylation reaction with a chlorinating agent to obtain the compound of formula (I): 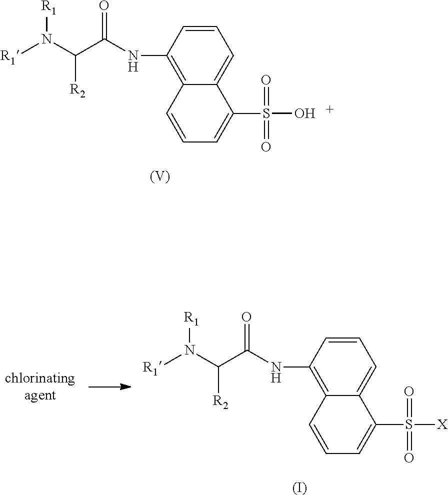
wherein, X is Cl, R₁ and R_{1′} are independently selected from C₁₋₇ alkyl, and R₂ is selected from H, C₁₋₇ alkyl, or benzyl.

16. The preparation method for the compound of formula (I) according to claim 15, wherein,
in the condensation reaction, the solvent is N,N-dimethylformamide (DMF); and/or
in the condensation reaction, the activating agent comprises one or more of 4-(4,6-dimethoxytriazin-2-yl)-4-methylmorpholine hydrochloride, 1-(3-dimethylaminopropyl)-3-ethylcarbodiimide hydrochloride and 1-hydroxybenzotriazole; and/or
in the condensation reaction, the base is an organic base; and/or
in the acylation reaction, the solvent comprises tetrahydrofuran and/or toluene; and/or
in the acylation reaction, the chlorinating agent comprises phosphorus pentachloride and/or acetyl chloride.

17. The preparation method for the compound of formula (I) according to claim 15, further comprising a process (1-1) or a process (1-2); wherein,
the process (1-1) comprises: subjecting a compound of formula (VI) to a reductive amination reaction with a compound of formula (A-1) and a compound of formula (A-2) in the presence of a reducing agent in a solvent to obtain the compound of formula (III): 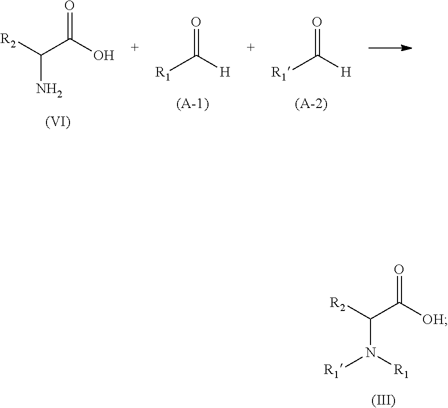
the process (1-2) comprises: subjecting a compound of formula (VI) to an alkylation reaction with a compound of formula (B-1) and a compound of formula (B-2) in the presence of a base in a solvent to obtain the compound of formula (III): 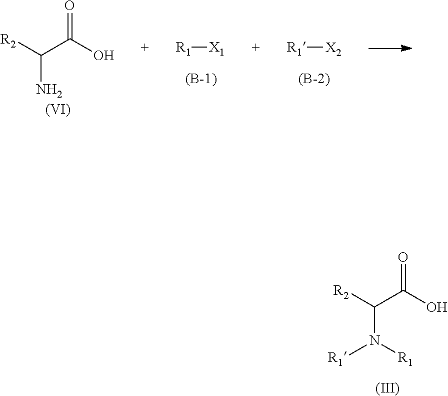
wherein X₁ and X₂ are independently halogen.

18. The preparation method for the compound of formula (I) according to claim 17, wherein,
in the reductive amination, the solvent reaction is methanol, acetonitrile, or a buffer of sodium acetate or phosphate with a pH of 2-12; and/or
in the reductive amination reaction, the reducing agent comprises sodium cyanoborohydride and/or 2-methylpyridine borane; and/or
in the alkylation reaction, the solvent is acetonitrile; and/or
in the alkylation reaction, the base is carbonate or bicarbonate.

19. The preparation method for the compound of formula (I) according to claim 18, wherein the carbonate is a potassium carbonate.

20. An isotope-labeled compound of formula (II) or a salt thereof: 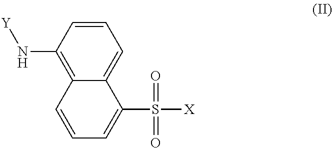
wherein Y is 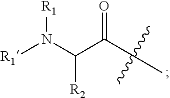
R₁ and R_{1′} are independently selected from C₁₋₇ alkyl;
R₂ is selected from H, C₁₋₇ alkyl, or benzyl;
X is selected from OH or halogen; and
at least one atom in Y is substituted by a heavier isotope thereof.

21. The isotope-labeled compound of formula (II) or the salt thereof according to claim 20, wherein,
at least one ¹H in Y is substituted by a heavier isotope ²H thereof, and/or
at least one ¹²C in Y is substituted by a heavier isotope ¹³C thereof; and/or
at least one ¹⁴N in Y is substituted by a heavier isotope ¹⁵N thereof, and/or
at least one ¹⁶O in Y is substituted by a heavier isotope ¹⁸O thereof.

22. The isotope-labeled compound of formula (II) or the salt thereof according to claim 21, wherein the isotope-labeled compound of formula (II) is any one of the following compounds: 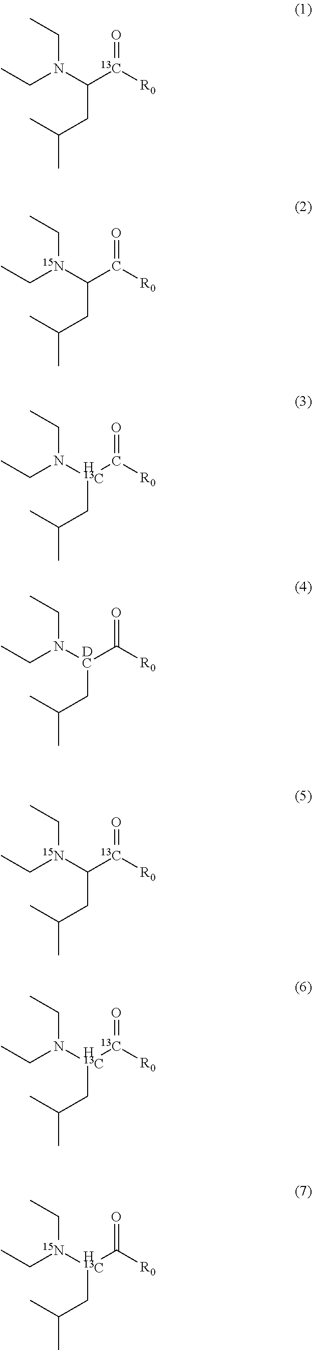 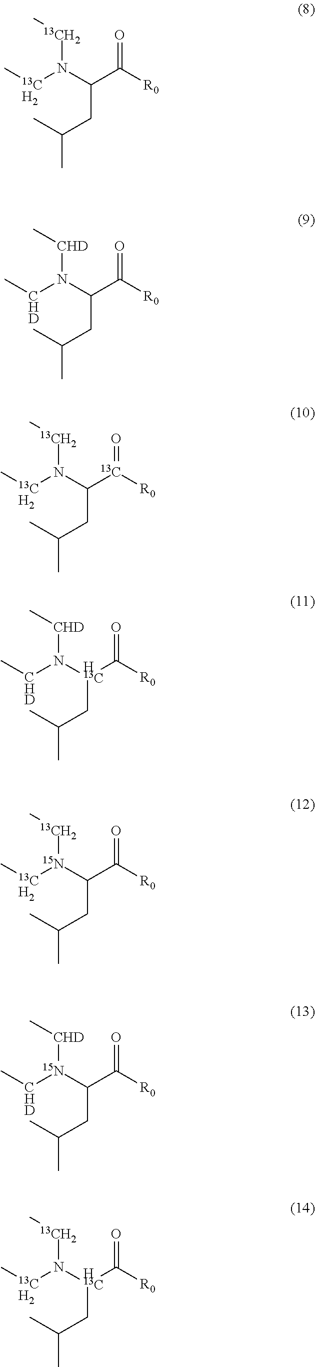 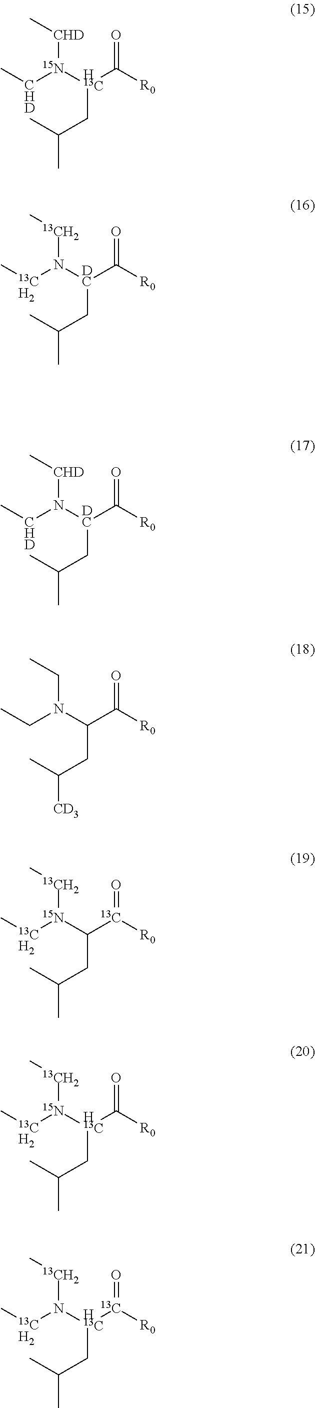 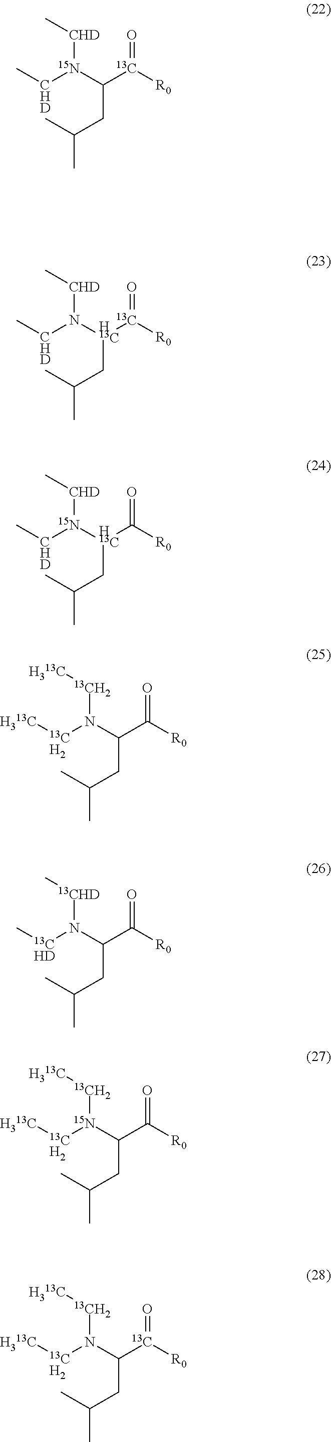 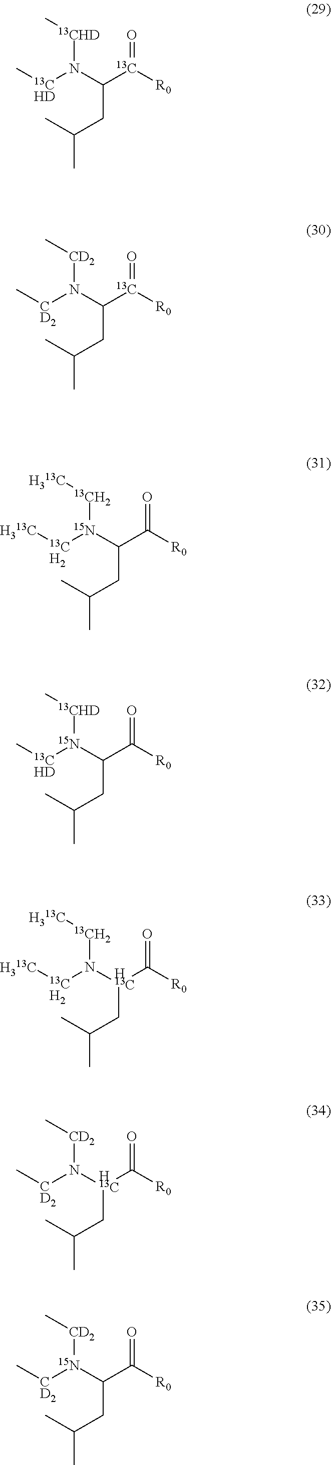 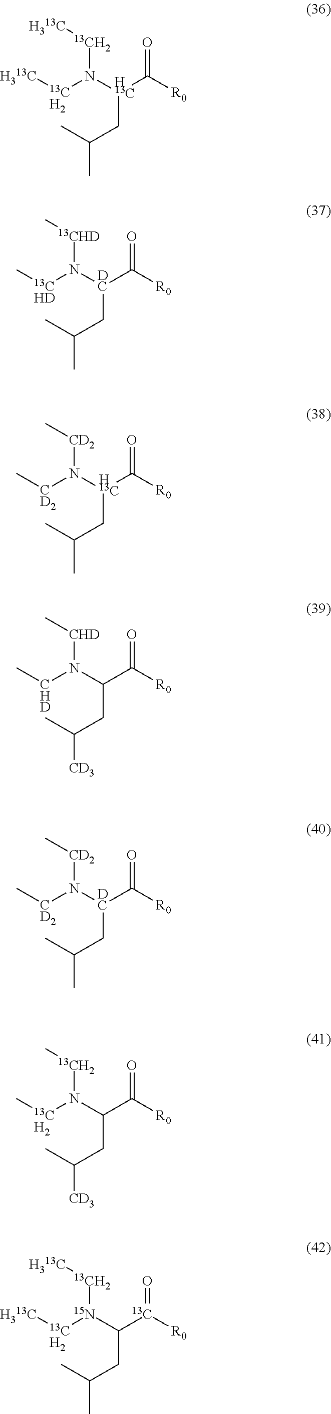 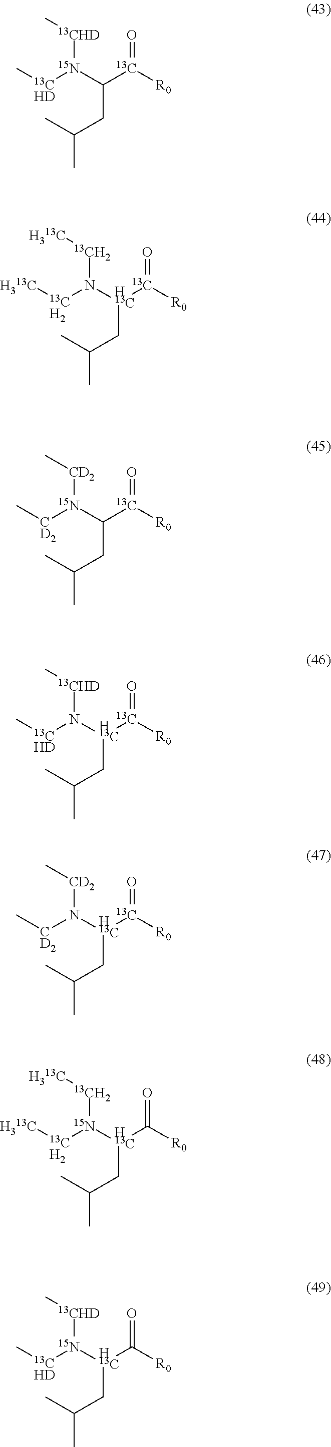 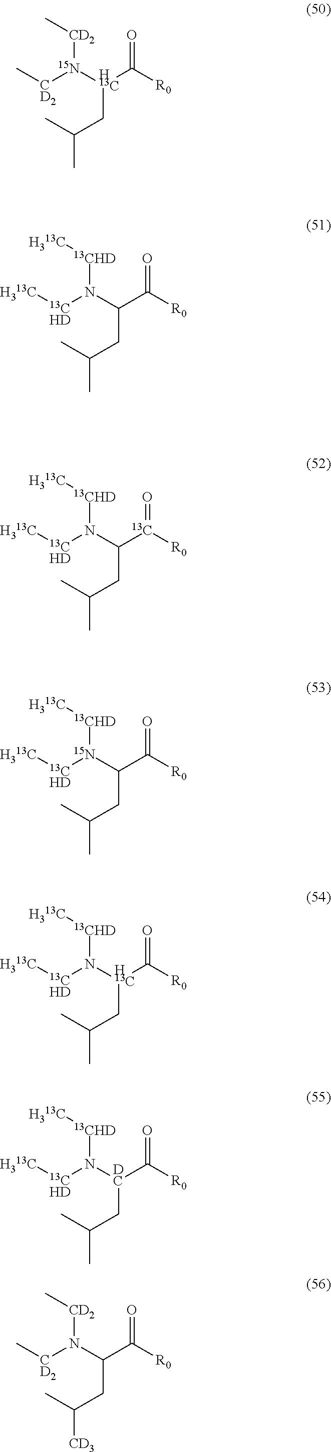 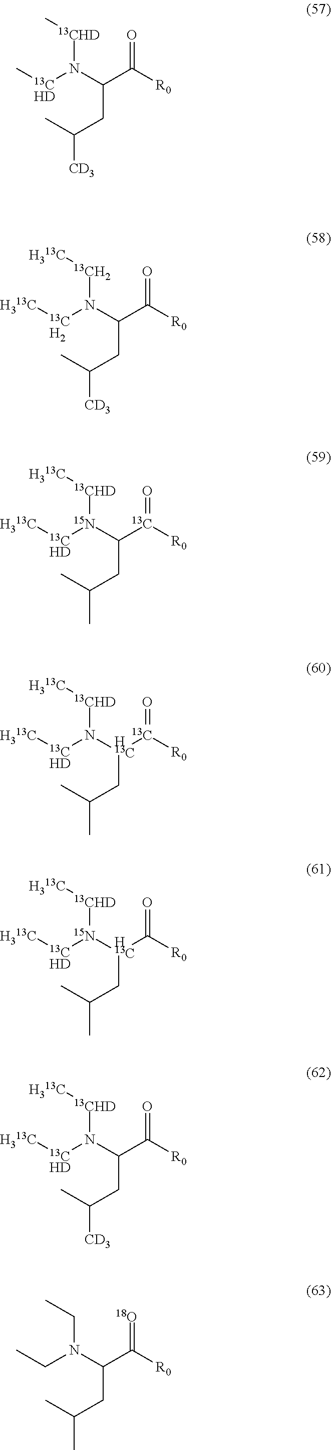 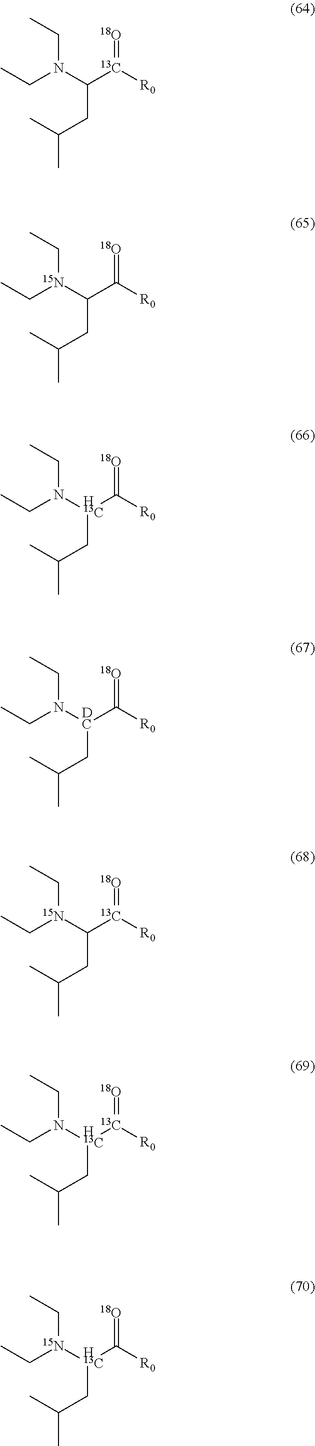 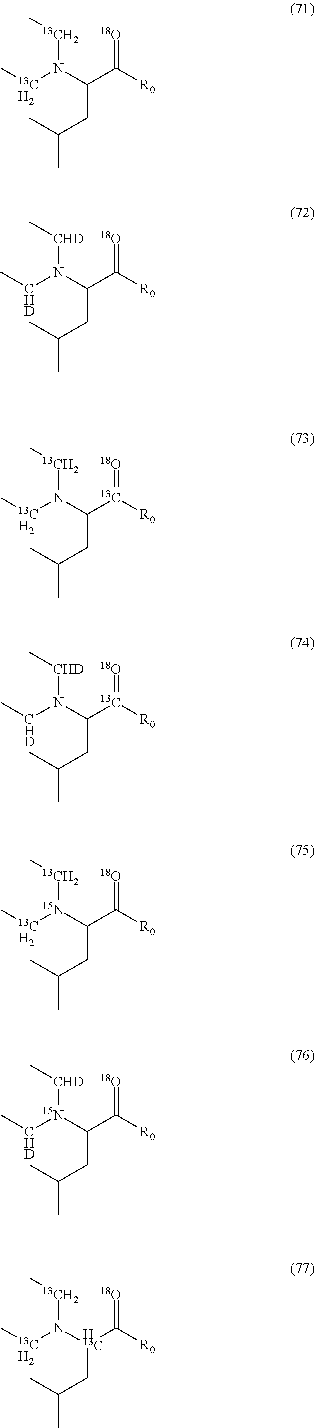 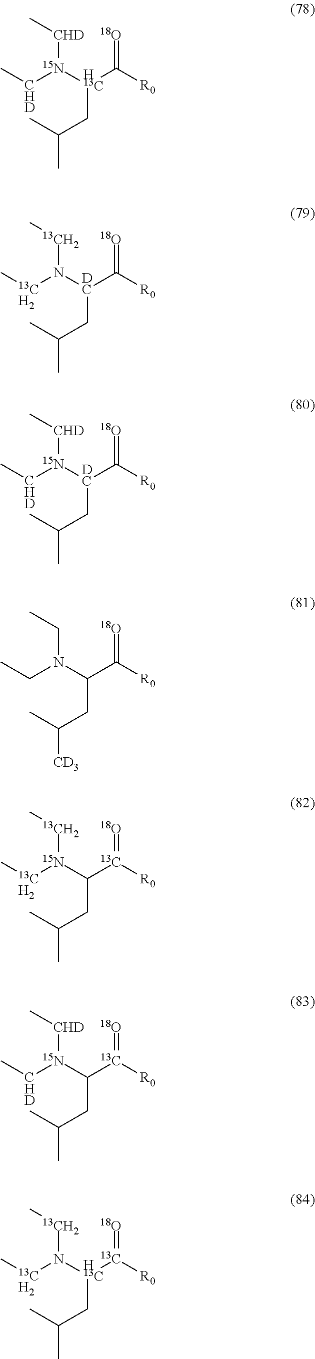 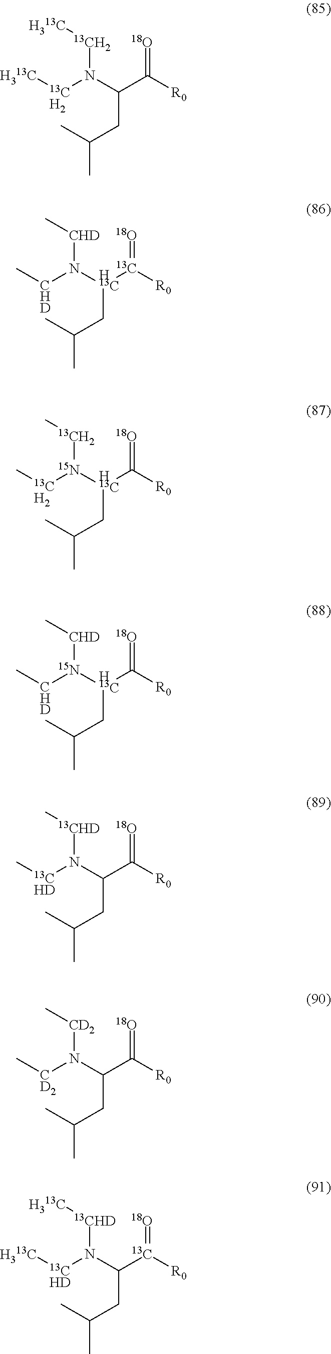 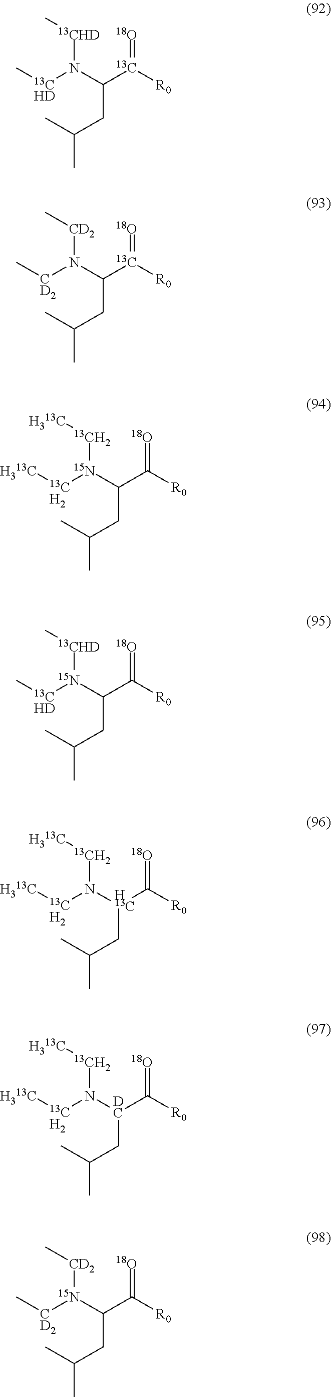 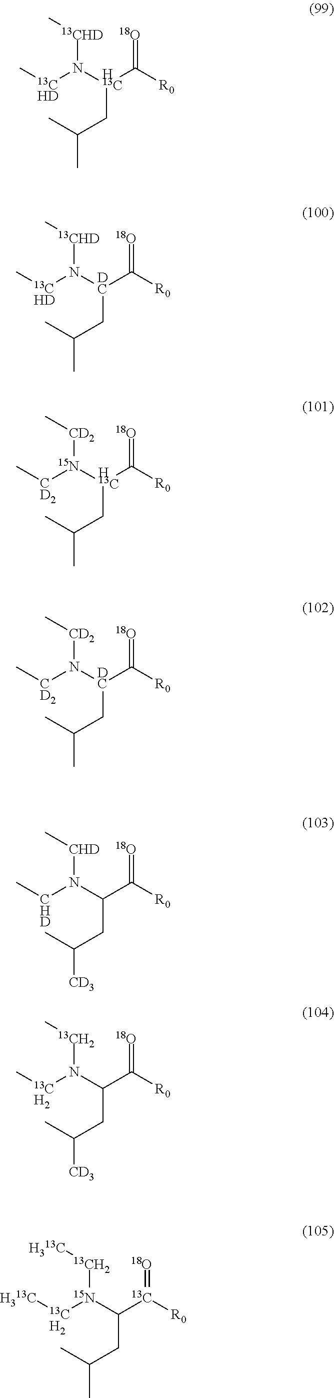 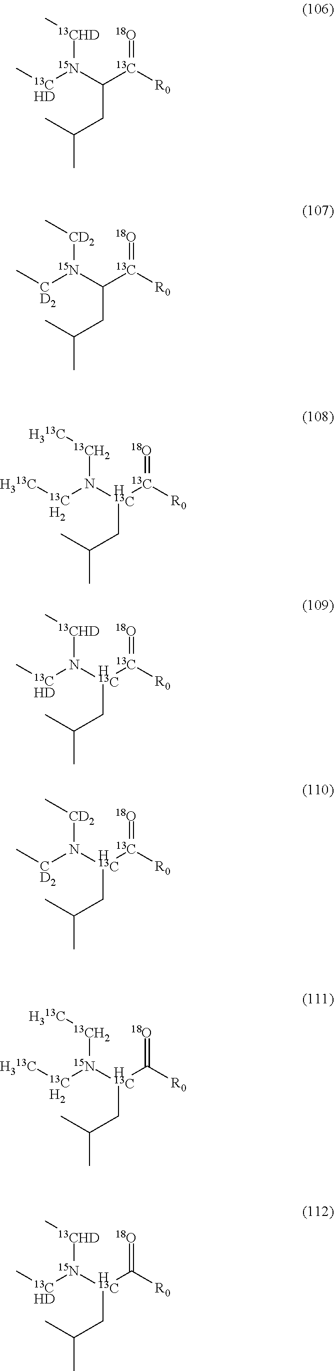 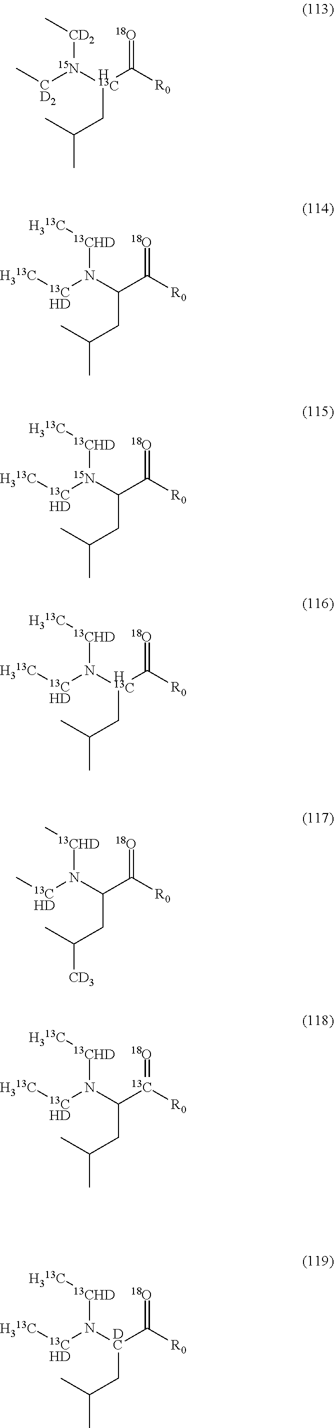 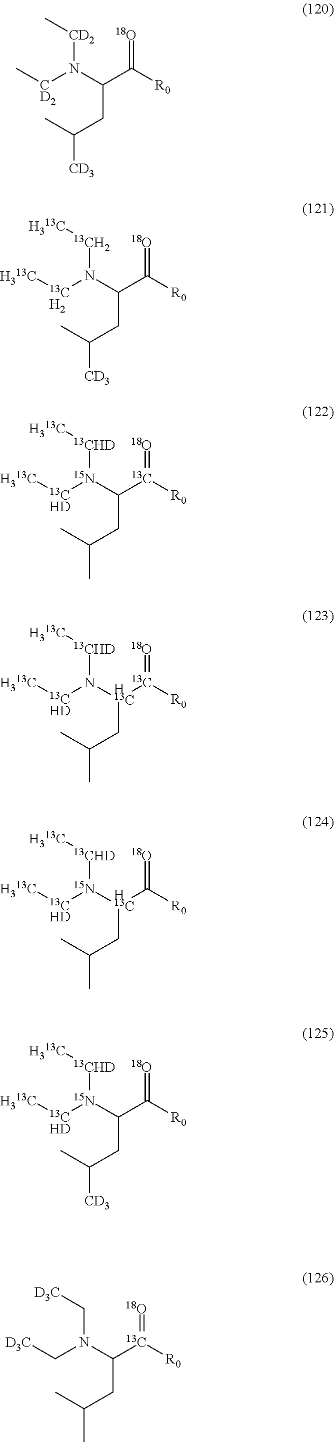 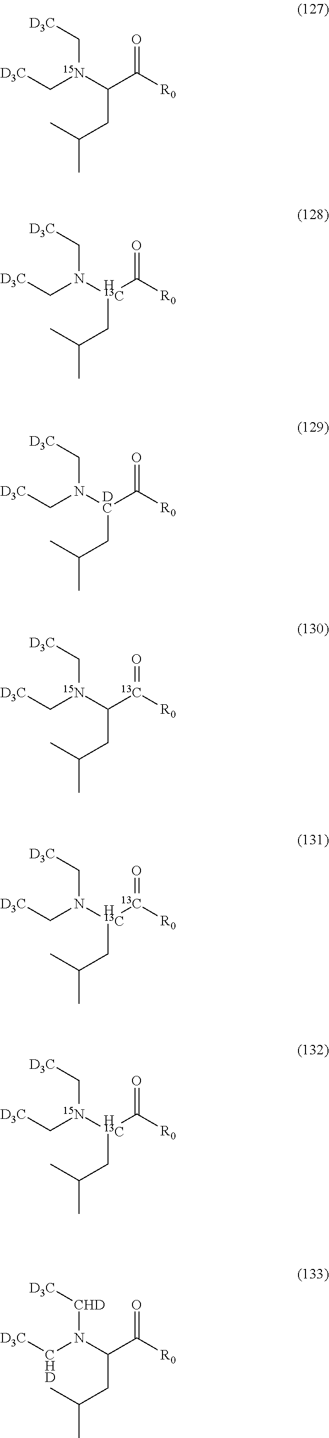 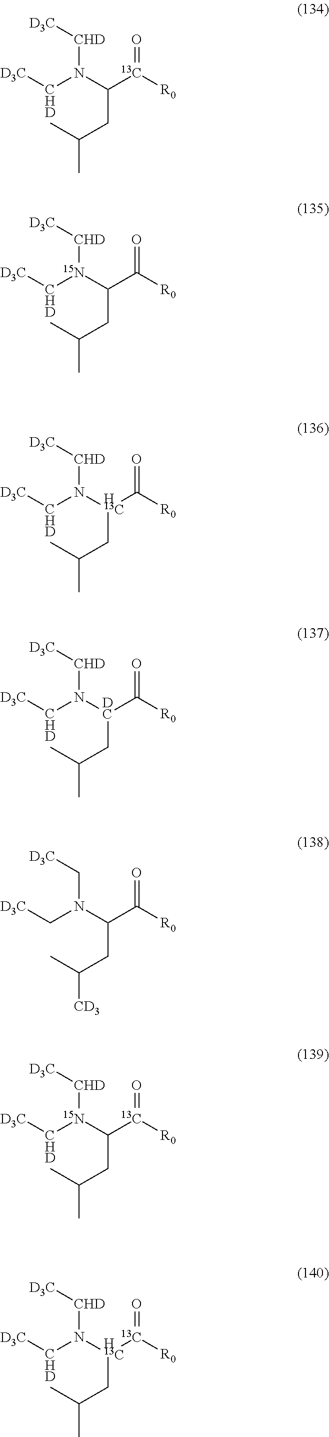 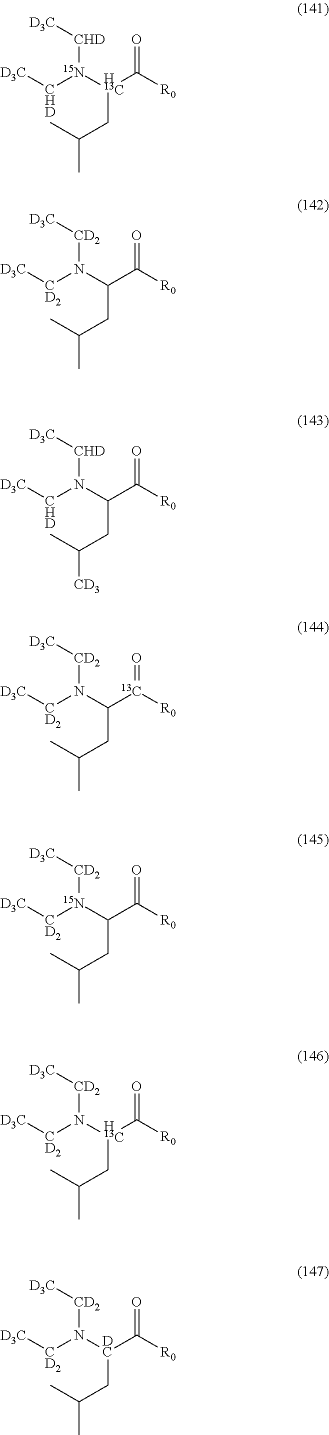 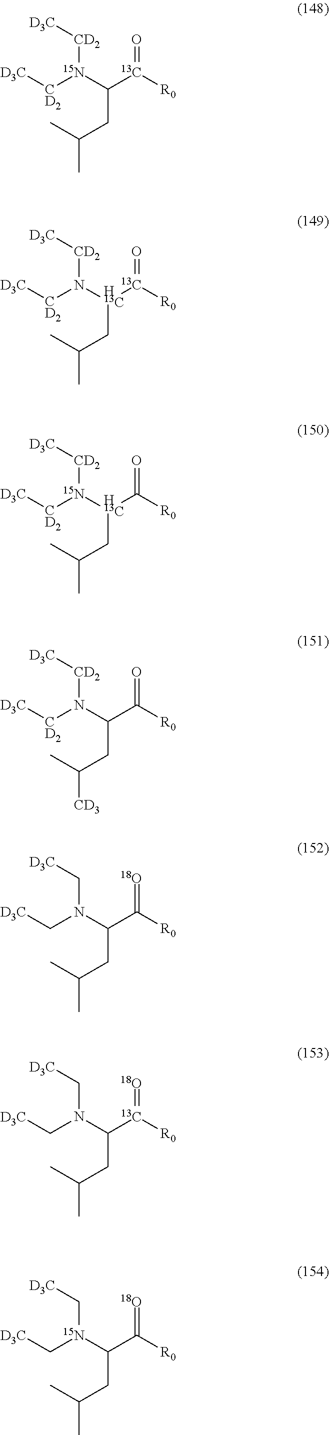 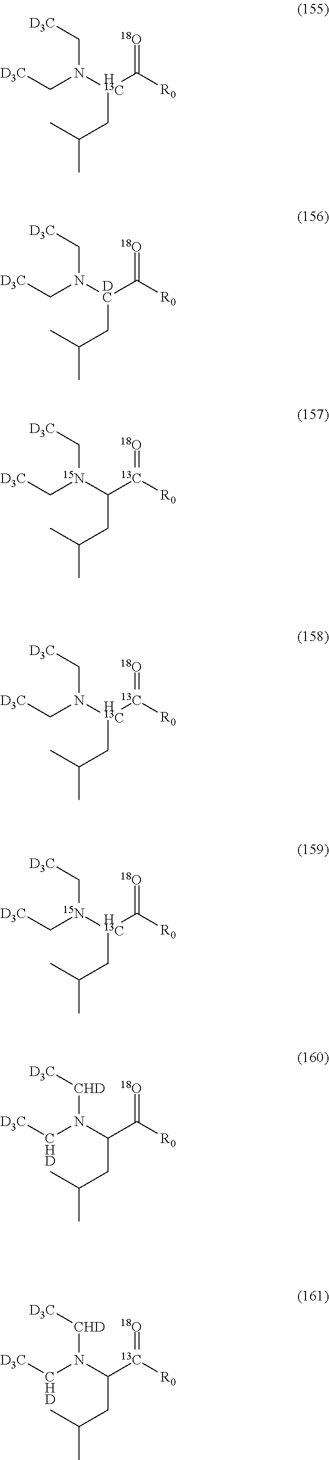 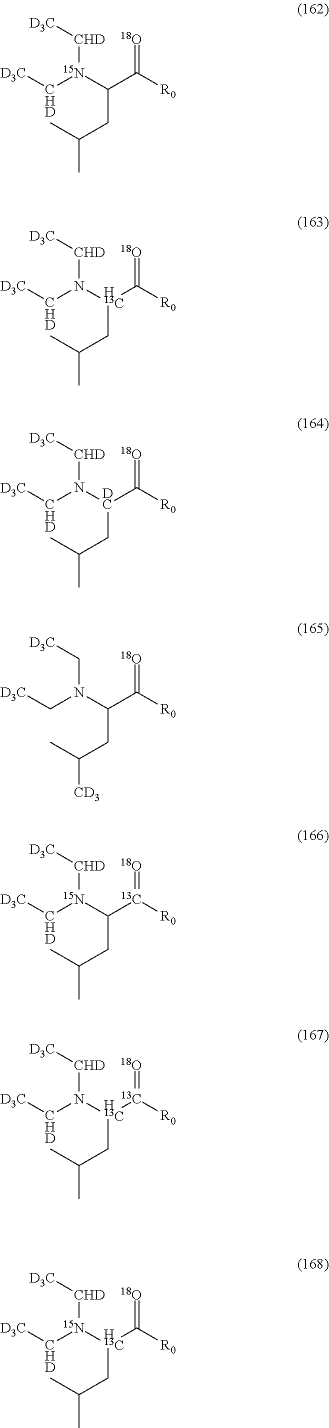 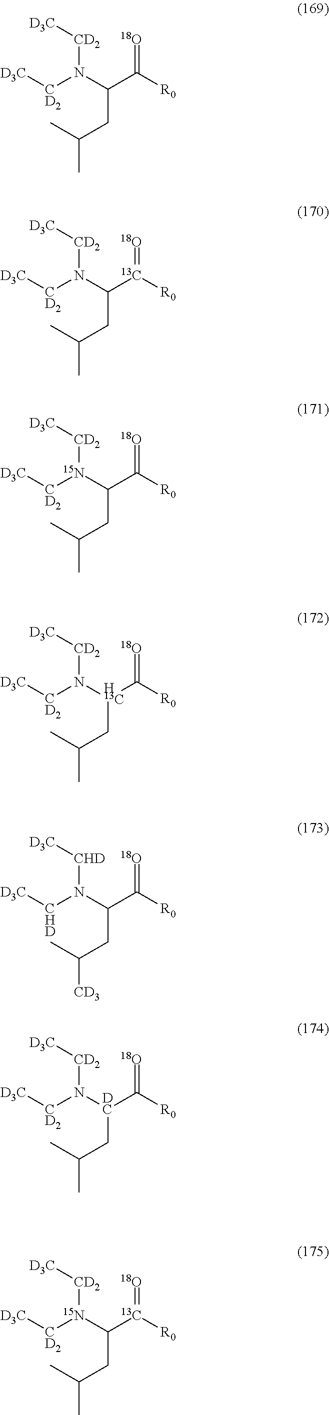 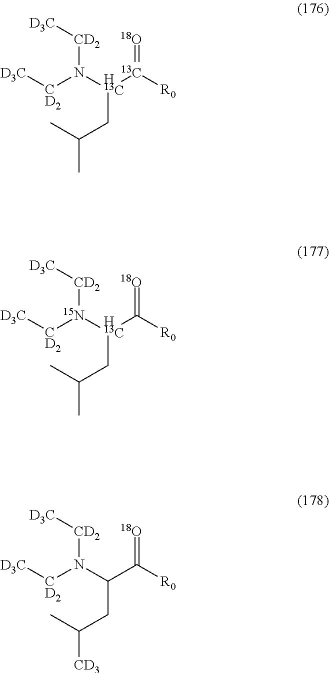
wherein R₀ is 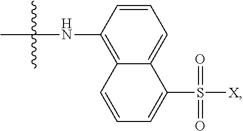
and X is OH or Cl.

23. Application of the compound of formula (I) or the salt thereof according to claim 1 as a derivatization reagent for detecting and/or separating compounds containing hydroxyl and/or amino groups.

24. Application of the isotope-labeled compound of formula (II) or the salt thereof according to claim 20 as a derivatization reagent for detecting and/or separating compounds containing hydroxyl and/or amino groups.
